(19) United States
(12) Patent Application Publication
(10) Pub. No.: US 2011/0183894 A1
(43) Pub. Date: Jul. 28, 2011

(54) ANTIVIRAL ACTIVITY OF THE PROTEIN SCYTOVIRIN AND METHODS OF USE

(76) Inventors: Barry O'Keefe, Frederick, MD (US); James B. Mcmahon, Frederick, MD (US); Kirk Gustafson, Frederick, MD (US); Yutaka Takebe, Tokyo (JP)

(21) Appl. No.: 13/056,892

(22) PCT Filed: Jul. 31, 2009
(86) PCT No.: PCT/US2009/004420

§ 371 Date: Apr. 12, 2011

Related U.S. Application Data
(60) Provisional application No. 61/137,511, filed on Jul. 31, 2008.

(51) Int. Cl. A61K 38/16; A61K 31/7088; A61K 48/00; C12N 7/06; A61P 31/12; A61P 31/14; A61P 31/18

(52) U.S. Cl. 514/3.8; 514/3.7; 514/44 R; 514/4.3; 435/238

(57) ABSTRACT

The present invention features methods of treating or preventing a viral infection in a subject, methods of inhibiting a virus in a biological sample, and methods of treating or preventing a viral infection caused by a virus in or on the skin or mucus membrane. The instant invention describes novel methods for treating viral infections, in particular infections caused by high mannose enveloped viruses, for example hepatitis C virus (HCV).

### CROSS-REFERENCE TO RELATED APPLICATION

This application claims the benefit of U.S. Provisional Application No. 61/137,511, which was filed Jul. 31, 2008, the entire contents of which are incorporated herein by reference.

### STATEMENT OF RIGHTS TO INVENTIONS MADE UNDER FEDERALLY SPONSORED RESEARCH

Research supporting this application was carried out by the United States of America as represented by the Secretary, Department of Health and Human Services. The Government has certain rights in this invention.

### INCORPORATION BY REFERENCE

Each of the applications and patents cited in this text, as well as each document or reference cited in each of the applications and patents (including during the prosecution of each issued patent; “application cited documents”), and each of the PCT and foreign applications or patents corresponding to and/or paragraphing priority from any of these applications and patents, and each of the documents cited or referenced in each of the application cited documents, are hereby expressly incorporated herein by reference. More generally, documents or references are cited in this text, either in a Reference List, or in the text itself; and, each of these documents or references (“herein-cited references”), as well as each document or reference cited in each of the herein-cited references (including any manufacturer's specifications, instructions, etc.), is hereby expressly incorporated herein by reference.

### BACKGROUND OF THE INVENTION

Hepatitis C virus (HCV) is a prevalent health problem with approximately 1% of the world's population infected with the virus. About 30,000 new cases of hepatitis C virus (HCV) infection are estimated to occur in the United States each year (Kolykhalov, A. A.; Mihalik, K.; Feinstone, S. M.; Rice, C. M.; 2000; J. Virol. 74: 2046-2051). HCV is not easily cleared by the hosts' immunological defenses; as many as 85% of the people infected with HCV become chronically infected. Many of these persistent infections result in chronic liver disease, including cirrhosis and hepatocellular carcinoma (Hoofnagle, J. H.; 1997; Hepatology 26: 15S-20S). HCV-associated end-stage liver disease is now the leading cause of liver transplantation. In the United States alone, hepatitis C is responsible for 8,000 to 10,000 deaths annually. Without effective intervention, the number is expected to triple in the next 10 to 20 years.

Currently, there is no vaccine to prevent HCV infection. The currently-utilized treatments for HCV are not fully effective and have serious complicating side effects that significantly reduce compliance. Prolonged treatment of chronically infected patients with interferon or interferon and ribavirin is the only currently approved therapy, but it achieves a sustained response in fewer than 50% of cases (Lindsay, K. L.; 1997; Hepatology 26: 71S-77S*, and Reichard, O.; Schvarcz, R.; Weiland, O.; 1997 Hepatology 26: 108S-111S*). Interferon treatment also induces severe side-effects (i.e. retinopathy, thyroiditis, acute pancreatitis, depression) that diminish the quality of life of treated patients. More recently, interferon in combination with ribavirin has been approved for patients non-responsive to IFN alone. However, the side effects caused by IFN are not alleviated with this combination therapy. Pegylated forms of interferons such as PEG-INTRON and PEGASYS can apparently partially address these deleterious side-effects but antiviral drugs still remain the avenue of choice for oral treatment of HCV.

HCV belongs to the family Flaviviridae, genus hepacivirus, which comprises three genera of small enveloped positive-strand RNA viruses (Rice, C. M.; 1996; “Flaviviridae: the viruses and their replication”; pp. 931-960 in Fields Virology; Fields, B. N.; Knipe, D. M.; Howley, P. M. (eds.); Lippincott-Raven Publishers, Philadelphia Pa. *). The 9.6 kb genome of HCV consists of a long open reading frame (ORF) flanked by 5′ and 3′ non-translated regions (NTR's). The HCV 5′ NTR is 341 nucleotides in length and functions as an internal ribosome entry site for cap-independent translation initiation (Lemon, S. H.; Honda, M.; 1997; Semin. Virol. 8: 274-288). The HCV polyprotein is cleaved co- and post-translationally into at least 10 individual polypeptides (Reed, K. E.; Rice, C. M.; 1999; Curr. Top. Microbiol. Immunol. 242: 55-84*). The structural proteins result from signal peptidases in the N-terminal portion of the polyprotein. Two viral proteases mediate downstream cleavages to produce non-structural (NS) proteins that function as components of the HCV RNA replicase. The NS2-3 protease spans the C-terminal half of the NS2 and the N-terminal one-third of NS3 and catalyses cis cleavage of the NS2/3 site. The same portion of NS3 also encodes the catalytic domain of the NS3-4A serine protease that cleaves at four downstream sites. The C-terminal two-thirds of NS3 is highly conserved amongst HCV isolates, with RNA-binding, RNA-stimulated NTPase, and RNA unwinding activities. Although NS4B and the NS5A phosphoprotein are also likely components of the replicase, their specific roles are unknown. The C-terminal polyprotein cleavage product, NS5B, is the elongation subunit of the HCV replicase possessing RNA-dependent RNA polymerase (RdRp) activity (Behrens, S. E.; Tomei, L.; DeFrancesco, R.; 1996; EMBO J. 15: 12-22; and Lohmann, V.; Korner, F.; Herian, U.; Bartenschlager, R.; 1997; J. Virol. 71: 8416-8428). It has been recently demonstrated that mutations destroying NS5B activity abolish infectivity of RNA in a chimp model (Kolykhalov, A. A.; Mihalik, K.; Feinstone, S. M.; Rice, C. M.; 2000; J. Virol. 74: 2046-2051).

Thus, a new therapeutic or adjunct therapy for HCV would fill a public health need. There is a need for improved HCV treatments which are more effective, and are not associated with the aforementioned disadvantages.

### SUMMARY OF THE INVENTION

It has now been demonstrated that the antiviral protein scytovirin (SVN) and the antiviral protein griffithsin (GRFT) both have potent (nanomolar) activity against Hepatitis C virus (HCV). The inventors of the instant application have developed novel compositions and methods for treating viral infections, in particular infections caused by high mannose enveloped viruses, for example HCV. The compositions can be used for the treatment or prevention of viral infections, for example HCV infection or HIV infection, or as an adjuvant to current therapies, or in methods of purification, for example as part of a dialysis system to remove virus particles from a subject, or to remove virus particles from biological fluids.

In a first aspect, the invention features a method of treating or preventing a viral infection in a subject comprising administering to the subject an effective amount of one or more of the following: (i) an isolated or purified antiviral protein comprising the amino acid sequence of SEQ ID NO: 1, an amino acid sequence that is about 90% or more identical to SEQ ID NO: 1, an amino acid sequence that is about 90% or more homologous to SEQ ID NO: 1, or a fragment thereof; (ii) an isolated or purified nucleic acid comprising a nucleotide sequence encoding the amino acid sequence of SEQ ID NO: 1; (iii) an isolated or purified antiviral protein comprising the amino acid sequence of SEQ ID NO: 2, an amino acid sequence that is about 90% or more identical to SEQ ID NO: 2, an amino acid sequence that is about 90% or more homologous to SEQ ID NO: 2, or a fragment thereof; (iv) an isolated or purified nucleic acid comprising a nucleotide sequence encoding the amino acid sequence of SEQ ID NO: 2; (v) an isolated or purified antiviral protein comprising the amino acid sequence of SEQ ID NO: 3, an amino acid sequence that is about 90% or more identical to SEQ ID NO: 3, an amino acid sequence that is about 90% or more homologous to SEQ ID NO: 3; (vi) an isolated or purified nucleic acid comprising a nucleotide sequence encoding the amino acid sequence of SEQ ID NO: 3, or a fragment thereof, thereby treating or preventing the viral infection in a subject.

SEQ ID NOs 1, 2 and 3 are set forth below:

| SEQ ID NO: 1 | |
|---|---|
| 1 | gsgptycwne annpggpnrc snnkqcdgar tcsssgfcqg tsrkpdpgpk gptycwdeak |
| 61 | npggpnrcsn skqcdgartc sssgfcqgta ghaaa |
| SEQ ID NO: 2 | |
| 1 | lgkfsqtcyn saiqgsvlts tcertnggyn tssidlnsvi envdgslkwq psnfietcrn |
| 61 | tqlagssela aecktraqqf vstkinlddh ianidgtlky e |
| SEQ ID NO: 3 | |
| 1 | slthrkfggs ggspfsglss iavrsgsyld xiiidgvhhg gsggnlsptf tfgsgeyisn |
| 61 | mtirsgdyid nisfetnmgr rfgpyggsgg santlsnvkv iqingsagdy ldsldiyyeq |
| 121 | y |

In one embodiment of the invention, the viral infection is caused by a virus with a coat protein comprising high-mannose oligosaccharides. In another embodiment, the virus is hepatitis C virus (HCV). In another embodiment, the virus is human immunodeficiency virus (HIV).

In another embodiment, the method further comprises a variant of (i) (ii) or (iii), wherein the variant comprises one or more conservative or neutral amino acid substitutions or one or more amino acid additions at the N-terminus or C-terminus, wherein the variant has antiviral activity characteristic of the antiviral protein consisting essentially of the amino acid sequence of SEQ ID NO: 1, SEQ ID NO: 2 or SEQ ID NO: 3.

In a related embodiment, the method further comprises a fusion protein of (i) (ii) or (iii) and at least one effector component, wherein the fusion protein has antiviral activity characteristic of the antiviral protein consisting essentially of the amino acid sequence of SEQ ID NO: 1, SEQ ID NO: 2 or SEQ ID NO: 3.

In still another embodiment, the fusion protein comprises albumin.

In a further embodiment of the invention, the nucleic acid comprising a nucleotide sequence encoding the amino acid sequence of SEQ ID NO: 1, SEQ ID NO: 2 or SEQ ID NO: 3 is contained in a vector. In a related embodiment, the vector is a retroviral, adenoviral, adeno-associated viral, or lentiviral vector. In another related embodiment, the vector comprises a promoter suitable for expression in a mammalian cell.

In another embodiment, the method of the invention as described herein further comprises the administration of one or more additional agents. In a related embodiment, the additional agent is selected from the group consisting of: antiviral agents, immunostimulants, and toxins. In another related embodiment, the one or more additional agents are administered prior to, simultaneously or subsequently to administration of the amino acid or nucleic acid of the above-described aspects.

In another aspect, the invention features a method of inhibiting a virus in a biological sample comprising contacting the biological sample with an effective amount of one or more of the following: (i) an isolated or purified antiviral protein comprising the amino acid sequence of SEQ ID NO: 1, an amino acid sequence that is about 90% or more identical to SEQ ID NO: 1, an amino acid sequence that is about 90% or more homologous to SEQ ID NO: 1, or a fragment thereof; (ii) an isolated or purified nucleic acid comprising a nucleotide sequence encoding the amino acid sequence of SEQ ID NO: 1; (iii) an isolated or purified antiviral protein comprising the amino acid sequence of SEQ ID NO: 2, an amino acid sequence that is about 90% or more identical to SEQ ID NO: 2, an amino acid sequence that is about 90% or more homologous to SEQ ID NO: 2, or a fragment thereof; (iv) an isolated or purified nucleic acid comprising a nucleotide sequence encoding the amino acid sequence of SEQ ID NO: 2; (v) an isolated or purified antiviral protein comprising the amino acid sequence of SEQ ID NO: 3, an amino acid sequence that is about 90% or more identical to SEQ ID NO: 3, an amino acid sequence that is about 90% or more homologous to SEQ ID NO: 3; (vi) an isolated or purified nucleic acid comprising a nucleotide sequence encoding the amino acid sequence of SEQ ID NO: 3, or a fragment thereof, and thereby inhibiting the virus in the biological sample.

In another aspect, the invention features a method of treating or preventing a viral infection caused by a virus in or on the skin or mucous membrane comprising: contacting the affected area with a topical composition comprising an effective amount of one or more of the following: (i) an isolated or purified antiviral protein comprising the amino acid sequence of SEQ ID NO: 1, an amino acid sequence that is about 90% or more identical to SEQ ID NO: 1, an amino acid sequence that is about 90% or more homologous to SEQ ID NO: 1, or a fragment thereof; (ii) an isolated or purified nucleic acid comprising a nucleotide sequence encoding the amino acid sequence of SEQ ID NO: 1; (iii) an isolated or purified antiviral protein comprising the amino acid sequence of SEQ ID NO: 2, an amino acid sequence that is about 90% or more identical to SEQ ID NO: 2, an amino acid sequence that is about 90% or more homologous to SEQ ID NO: 2, or a fragment thereof; (iv) an isolated or purified nucleic acid comprising a nucleotide sequence encoding the amino acid sequence of SEQ ID NO: 2; (v) an isolated or purified antiviral protein comprising the amino acid sequence of SEQ ID NO: 3, an amino acid sequence that is about 90% or more identical to SEQ ID NO: 3, an amino acid sequence that is about 90% or more homologous to SEQ ID NO: 3; (vi) an isolated or purified nucleic acid comprising a nucleotide sequence encoding the amino acid sequence of SEQ ID NO: 3, or a fragment thereof, thereby treating or preventing a viral infection caused by a virus in or on the skin or mucous membrane.

In one embodiment, the viral infection is caused by a virus having a coat protein comprising high-mannose oligosaccharides. In another embodiment, the virus is HCV. In another embodiment, the virus is HIV.

In one embodiment, the topical composition is a foam or a gel.

In another aspect, the invention features a method of inhibiting a virus in or on an object comprising contacting the object with an effective amount of one or more of the following: (i) an isolated or purified antiviral protein comprising the amino acid sequence of SEQ ID NO: 1, an amino acid sequence that is about 90% or more identical to SEQ ID NO: 1, an amino acid sequence that is about 90% or more homologous to SEQ ID NO: 1, or a fragment thereof; (ii) an isolated or purified nucleic acid comprising a nucleotide sequence encoding the amino acid sequence of SEQ ID NO: 1; (iii) an isolated or purified antiviral protein comprising the amino acid sequence of SEQ ID NO: 2, an amino acid sequence that is about 90% or more identical to SEQ ID NO: 2, an amino acid sequence that is about 90% or more homologous to SEQ ID NO: 2, or a fragment thereof; (iv) an isolated or purified nucleic acid comprising a nucleotide sequence encoding the amino acid sequence of SEQ ID NO: 2; (v) an isolated or purified antiviral protein comprising the amino acid sequence of SEQ ID NO: 3, an amino acid sequence that is about 90% or more identical to SEQ ID NO: 3, an amino acid sequence that is about 90% or more homologous to SEQ ID NO: 3; (vi) an isolated or purified nucleic acid comprising a nucleotide sequence encoding the amino acid sequence of SEQ ID NO: 3, or a fragment thereof, thereby inhibiting the virus in or on the object.

In one embodiment, the biological sample is selected from the group consisting of: blood, a blood product, cells, a tissue, an organ, sperm, a vaccine formulation, and a bodily fluid.

In another aspect, the invention features a method for elimination of a virus from the blood of a subject comprising contacting the blood with an effective amount of one or more of the following: (i) an isolated or purified antiviral protein comprising the amino acid sequence of SEQ ID NO: 1, an amino acid sequence that is about 90% or more identical to SEQ ID NO: 1, an amino acid sequence that is about 90% or more homologous to SEQ ID NO: 1, or a fragment thereof; (ii) an isolated or purified nucleic acid comprising a nucleotide sequence encoding the amino acid sequence of SEQ ID NO: 1; (iii) an isolated or purified antiviral protein comprising the amino acid sequence of SEQ ID NO: 2, an amino acid sequence that is about 90% or more identical to SEQ ID NO: 2, an amino acid sequence that is about 90% or more homologous to SEQ ID NO: 2, or a fragment thereof; (iv) an isolated or purified nucleic acid comprising a nucleotide sequence encoding the amino acid sequence of SEQ ID NO: 2; (v) an isolated or purified antiviral protein comprising the amino acid sequence of SEQ ID NO: 3, an amino acid sequence that is about 90% or more identical to SEQ ID NO: 3, an amino acid sequence that is about 90% or more homologous to SEQ ID NO: 3; (vi) an isolated or purified nucleic acid comprising a nucleotide sequence encoding the amino acid sequence of SEQ ID NO: 3, or a fragment thereof, thereby eliminating the virus from the blood.

In one embodiment, the object is a solution, a medical supply, or a medical equipment.

In another embodiment of the above aspects, the virus has a coat protein comprising high-mannose oligosaccharides. In a further related embodiment, the virus is hepatitis C virus (HCV). In another further embodiment, the virus is human immunodeficiency virus (HIV).

In one embodiment of any one of the above-mentioned aspects, the method further comprises a variant of (i) (ii) or (iii), wherein the variant comprises one or more conservative or neutral amino acid substitutions or one or more amino acid additions at the N-terminus or C-terminus, wherein the variant has antiviral activity characteristic of the antiviral protein consisting essentially of the amino acid sequence of SEQ ID NO: 1, SEQ ID NO: 2 or SEQ ID NO: 3.

In another embodiment of any one of the above-mentioned aspects, the method further comprises a fusion protein of (i) (ii) or (iii) and at least one effector component, wherein the fusion protein has antiviral activity characteristic of the antiviral protein consisting essentially of the amino acid sequence of SEQ ID NO: 1, SEQ ID NO: 2 or SEQ ID NO: 3.

In one embodiment, the fusion protein comprises albumin.

In another embodiment of any one of the above-mentioned aspects, the method further comprises the administration of one or more additional agents. In a related embodiment, the additional agents are selected from the group consisting of: antiviral agents, immunostimulants, and toxins.

In another aspect, the invention features a method of treating or preventing a viral infection in a subject comprising administering to the subject one or more antibodies selected from: (i) an antibody that binds a protein comprising the amino acid sequence of SEQ ID NO: 1; (ii) an antibody that binds a protein comprising the amino acid sequence of SEQ ID NO: 2; (iii) an antibody that binds a protein comprising the amino acid sequence of SEQ ID NO: 3, or a fragment thereof, in an amount sufficient to induce in the subject an immune response to the virus; and thereby treating or preventing the viral infection in a subject.

In one embodiment, the viral infection is caused by a virus with a coat protein comprising high-mannose oligosaccharides. In another embodiment, the virus is hepatitis C virus (HCV). In another embodiment, the virus is human immunodeficiency virus (HIV).

In another embodiment, the method further comprises the administration of one or more additional agents. In a further embodiment, the additional agents are selected from the group consisting of: antiviral agents, immunostimulants, and toxins.

In another aspect, the invention features a method of inhibiting a virus in a biological sample comprising administering to the subject one or more antibodies selected from: (i) an antibody that binds a protein comprising the amino acid sequence of SEQ ID NO: 1; (ii) an antibody that binds a protein comprising the amino acid sequence of SEQ ID NO: 2; (iii) an antibody that binds a protein comprising the amino acid sequence of SEQ ID NO: 3, or a fragment thereof, in an amount sufficient to induce in the subject an immune response to the virus; thereby inhibiting the virus in a biological sample.

In one embodiment, the biological sample is selected from the group consisting of: blood, a blood product, cells, a tissue, an organ, sperm, a vaccine formulation, and a bodily fluid.

In another aspect, the invention features a method for elimination of a virus from the blood of a subject comprising administering to the subject one or more antibodies selected from: (i) an antibody that binds a protein comprising the amino acid sequence of SEQ ID NO: 1; (ii) an antibody that binds a protein comprising the amino acid sequence of SEQ ID NO: 2; (iii) an antibody that binds a protein comprising the amino acid sequence of SEQ ID NO: 3, or a fragment thereof, in an amount sufficient to induce in the subject an immune response to the virus; thereby eliminating the virus from the blood.

In one embodiment, the blood is from a blood transfusion.

In another aspect, the invention features a method of treating or preventing a viral infection caused by a virus in or on the skin or mucous membrane comprising administering to the subject one or more antibodies selected from: (i) an antibody that binds a protein comprising the amino acid sequence of SEQ ID NO: 1; (ii) an antibody that binds a protein comprising the amino acid sequence of SEQ ID NO: 2; (iii) an antibody that binds a protein comprising the amino acid sequence of SEQ ID NO: 3, or a fragment thereof, in an amount sufficient to induce in the subject an immune response to the virus; thereby treating or preventing a viral infection caused by a virus in or on the skin or mucous membrane.

In one embodiment, the topical composition is a foam or a gel.

In another aspect, the invention features a method of inhibiting a virus in or on an object comprising administering to the subject one or more antibodies selected from: (i) an antibody that binds a protein comprising the amino acid sequence of SEQ ID NO: 1; (ii) an antibody that binds a protein comprising the amino acid sequence of SEQ ID NO: 2; (iii) an antibody that binds a protein comprising the amino acid sequence of SEQ ID NO: 3, or a fragment thereof, in an amount sufficient to induce in the subject an immune response to the virus; thereby inhibiting the virus in or on an object.

In one embodiment, the object is a solution, a medical supply, or a medical equipment.

In another embodiment of any one of the above aspects, the virus has a coat protein comprising high-mannose oligosaccharides. In another embodiment, the virus is hepatitis C virus (HCV). In another embodiment, the virus is human immunodeficiency virus (HIV).

In another embodiment of any one of the above aspects, the isolated or purified antiviral protein comprising the amino acid sequence of SEQ ID NO: 1, 2, or 3, or a fragment thereof is administered at a concentration of 5-250 ng/ml.

In another embodiment of any one of the above aspects, the subject is a human.

Other features and advantages of the invention will be apparent from the detailed description, and from the claims.

### BRIEF DESCRIPTION OF THE DRAWINGS

FIG. 1 (A-C) is a panel of three graphs showing the activity of cyanovirin (A), scytovirin (B), or griffithsin (C) against the hepatitis C virus (HCV).

FIG. 2 shows the sequences of SEQ ID NO: 1, SEQ ID NO: 2 and SEQ ID NO: 3.

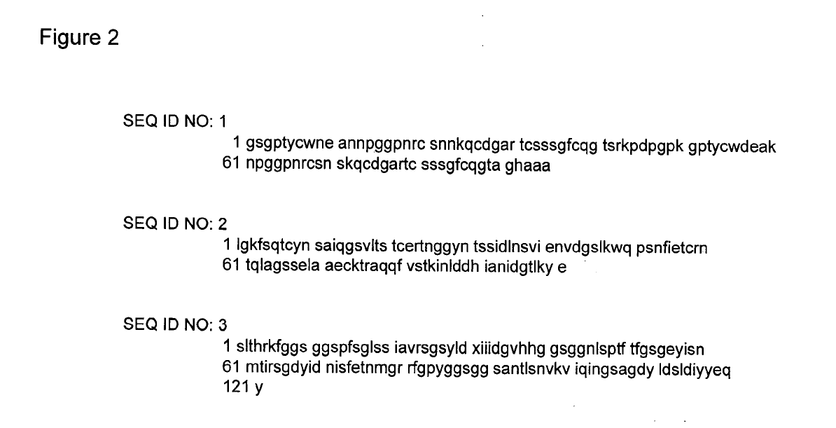

### DETAILED DESCRIPTION

The instant invention is based upon the finding that the antiviral protein scytovirin (SVN) has been found to have potent activity against the hepatitis C virus (HCV). The instant invention describes novel methods for treating viral infections, in particular infections caused by high mannose enveloped viruses, for example hepatitis C virus (HCV).

### DEFINITIONS

The following definitions are provided for specific terms which are used in the following written description.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used in the specification and claims, the singular form “a”, “an” and “the” include plural references unless the context clearly dictates otherwise.

In this disclosure, “comprises,” “comprising,” “containing” and “having” and the like can have the meaning ascribed to them in U.S. patent law and can mean “includes,” “including,” and the like; “consisting essentially of or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments. The terms “administration” or “administering” are defined to include an act of providing a compound or pharmaceutical composition of the invention to a subject in need of treatment.

The phrase “in combination with” is intended to refer to all forms of administration that provide the inhibitory nucleic acid molecule and the chemotherapeutic agent together, and can include sequential administration, in any order.

The terms “polypeptide” and “protein” or protein as used herein are meant to refer to a polymer of amino acid residues and are not limited to a minimum length of the product. Thus, peptides, oligopeptides, dimers, multimers, and the like, are included within the definition. Both full-length proteins and fragments thereof are encompassed by the definition. The terms also include postexpression modifications of the polypeptide, for example, glycosylation, acetylation, phosphorylation and the like. Furthermore, for purposes of the present invention, a “polypeptide” refers to a protein which includes modifications, such as deletions, additions and substitutions (generally conservative in nature), to the native sequence, so long as the protein maintains the desired activity. These modifications may be deliberate, as through site-directed mutagenesis, or may be accidental, such as through mutations of hosts which produce the proteins or errors due to PCR amplification.

The term “scytovirin” (SVN) as used herein are meant to refer to an isolated or purified protein consisting essentially of SEQ ID NO: 1, as well as antiviral fragments thereof, whether isolated or purified from nature, recombinantly produced, or synthesized, and substantially identical or homologous proteins (as defined herein). An antiviral fragment can be generated, for example, by removing 1-20, preferably 1-10, more preferably 1, 2, 3, 4, or 5, and most preferably 1 or 2, amino acids from one or both ends, preferably from only one end, and most preferably from the amino-terminal end, of the wild-type scytovirin, such as wild-type scytovirin of SEQ ID NO: 1.

The term “cyanovirin” (CV-N) as used herein is meant to refer to an isolated or purified protein consisting essentially of SEQ ID NO: 2, as well as antiviral fragments thereof, whether isolated or purified from nature, recombinantly produced, or synthesized, and substantially identical or homologous proteins (as defined herein). An antiviral fragment can be generated, for example, by removing 1-20, preferably 1-10, more preferably 1, 2, 3, 4, or 5, and most preferably 1 or 2, amino acids from one or both ends, preferably from only one end, and most preferably from the amino-terminal end, of the wild-type cyanovirin, such as wild-type cyanovirin of SEQ ID NO: 2.

The term “griffithsin” (GRFT) as used herein is meant to refer to an isolated or purified protein consisting essentially of SEQ ID NO: 3, as well as antiviral fragments thereof, whether isolated or purified from nature, recombinantly produced, or synthesized, and substantially identical or homologous proteins (as defined herein). An antiviral fragment can be generated, for example, by removing 1-20, preferably 1-10, more preferably 1, 2, 3, 4, or 5, and most preferably 1 or 2, amino acids from one or both ends, preferably from only one end, and most preferably from the amino-terminal end, of the wild-type griffithsin, such as wild-type griffithsin of SEQ ID NO: 3.

The term “mucous membranes,” “mucosal membranes,” and “mucosal tissue” are used interchangeably and refer to the surfaces of the nasal (including anterior nares, nasopharangyl cavity, etc.), oral (e.g., mouth including the inner lip, buccal cavity and gums), vaginal, and other similar tissues.

The term “fragment” as used herein is meant to include a polypeptide consisting of only a part of the intact full-length polypeptide sequence and structure. The fragment can include a C-terminal deletion and/or an N-terminal deletion of the native polypeptide. An “immunogenic fragment” or “antigenic fragment” of a particular protein, e.g., will generally include at least about 5-10 contiguous amino acid residues of the full-length molecule, preferably at least about 15-25 contiguous amino acid residues of the full-length molecule, and most preferably at least about 20-50 or more contiguous amino acid residues of the full-length molecule, that define an epitope, or any integer between 5 amino acids and the full-length sequence, provided that the fragment in question retains immunogenic or antigenic activity, as measured by the assays described herein or any standard assay known in the art.

The term “antiviral agent” as used herein in meant to include an agent (compound or biological) that is effective to inhibit the formation and/or replication of a virus in a mammal. This includes agents that interfere with either host or viral mechanisms necessary for the formation and/or replication of a virus in a mammal. Antiviral agents include, for example, ribavirin, amantadine, VX-497 (merimepodib, Vertex Pharmaceuticals), VX-498 (Vertex Pharmaceuticals), Levovirin, Viramidine, Ceplene (maxamine), XTL-001 and XTL-002 (XTL Biopharmaceuticals).

As used herein, the term “treating” or “treat” is meant to refer to the administration of a compound or composition according to the present invention to alleviate or eliminate symptoms of the viral infection in the subject and/or to reduce viral load in the subject. In certain examples, treating is meant to refer to alleviating or eliminating symptoms of HCV in the subject, and/or to reduce the viral load in the subject.

As used herein, the term “preventing” or “prevent” is meant to refer to the administration of a compound or composition according to the present invention post-exposure of the individual to the virus but before the appearance of symptoms of the disease, and/or prior to the detection of the virus in the blood. In certain examples, prevention is meant to refer to prevention of HCV.

A “nucleic acid” molecule or “polynucleotide” can include both double- and single-stranded sequences and refers to, but is not limited to, cDNA from viral, prokaryotic or eukaryotic mRNA, genomic DNA sequences from viral (e.g. DNA viruses and retroviruses) or prokaryotic DNA, and especially synthetic DNA sequences. The term also captures sequences that include any of the known base analogs of DNA and RNA.

A “coding sequence” or a sequence which “encodes” a selected polypeptide, is a nucleic acid molecule which is transcribed (in the case of DNA) and translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A transcription termination sequence may be located 3′ to the coding sequence.

The term “homology” as used herein is meant to refer to the percent identity between two polynucleotide or two polypeptide moieties. Two DNA, or two polypeptide sequences are “substantially homologous” to each other when the sequences exhibit at least about 50%, preferably at least about 75%, more preferably at least about 80%-85%, preferably at least about 90%, and most preferably at least about 95%-98%, or more, sequence identity over a defined length of the molecules. As used herein, substantially homologous also refers to sequences showing complete identity to the specified DNA or polypeptide sequence.

The term “identity” or “identical” as used herein refers to an exact nucleotide-to-nucleotide or amino acid-to-amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353-358, National biomedical Research Foundation, Washington, D.C., which adapts the local homology algorithm of Smith and Waterman Advances in Appl. Math. 2:482-489, 1981 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Another method of establishing percent identity is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the following internet address: http://www.ncbi.nln.gov/cgi-bin/BLAST.

Alternatively, homology can be determined by hybridization of polynucleotides under conditions which form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

### Scytovirin (SVN)

SVN is a lectin isolated from cyanobacterium Scytonema varium. A single chain of SVN contains 95 amino acids; ten of them, which are cysteines, form five intrachain disulfide bonds. Their pattern, elucidated by mass spectrometry of fragments obtained by trypsin digests, was shown to be C7-C55, C20-C26, C32-C38, C68-C74, and C80-C86 (Bokesch et al. 2003 A potent novel antiHIV protein from the cultured cyanobacterium Scytonema varium. Biochemistry 42: 2578-2584). SVN demonstrates internal sequence duplication, suggesting the presence of two functional domains linked by the C7-C55 disulfide bond. The extent of identity of the sequences of the N-terminal part of the molecule (residues 1-48) and the C-terminal part (residues 49-95) is very high (75%).

SVN binds to glycosylated gp160, gp120, and gp41 and interacts with oligosaccharides, specifically α1-2, α1-2, α1-6 linked tetrasaccharide units, but with no reported binding to α1-2, α1-2 linked trisaccharides (Adams et al., 2003. Encoded fiber-optic microsphere arrays for probing protein-carbohydrate interactions. Angew Chem Int Ed Engl 42: 5317-5320.). Although it does not show significant specificity for mannose or N-acetylglucosamine, its binding to gp120 can be inhibited by Man8 GlcNAc2 or Man9 GlcNAc2. SVN displays nanomolar activity against T-tropic strains and primary isolates of HIV-1, appearing to be a good inhibitor of HIV binding and/or fusion (Bokesch et al., 2003).

The primary structure of SVN exhibits 55% similarity to the chitin-binding domain of Volvox carteri lectin and a slightly lower level of similarity to the sequence of lectin from Urtica dioica (UDA). A synthetic gene encoding SVN has been constructed and expressed in E. coli. The recombinant protein was found to have correct disulfide-bonding pattern and exhibit both gp160-binding activity and antiHIV activity.

In certain embodiments, the term “scytovirin” (SVN) as used herein are meant to refer to an isolated or purified protein consisting essentially of SEQ ID NO: 1, as well as antiviral fragments thereof, whether isolated or purified from nature, recombinantly produced, or synthesized, and substantially identical or homologous proteins (as defined herein). An antiviral fragment can be generated, for example, by removing 1-20, preferably 1-10, more preferably 1, 2, 3, 4, or 5, and most preferably 1 or 2, amino acids from one or both ends, preferably from only one end, and most preferably from the amino-terminal end, of the wild-type scytovirin, such as wild-type scytovirin of SEQ ID NO: 1. SEQ ID NO: 1 is shown below, and corresponds to NCBI Accession No. 2QT4_A.

| SEQ ID NO: 1 | |
|---|---|
| 1 | gsgptycwne annpggpnrc snnkqcdgar tesssgfcqg tsrkpdpgpk gptycwdeak |
| 61 | npggpnrcsn skqcdgartc sssgfcqgta ghaaa |

### Cyanovirin-N (CV-N)

Cyanovirin-N (CV-N) is a lectin, and a potent HIV-inactivating protein that was originally isolated and identified from aqueous extracts of the cultured cyanobacterium Nostoc ellipsosporum (U.S. Pat. No. 6,420,336, incorporated by reference in its entirety herein), and was identified in a screening effort aimed at the discovery of new sources of HIV inhibitors (Boyd, M. R. In AIDS, etiology, diagnosis, treatment and prevention. (DeVita, V. R., Hellman, S. & Rosenberg, S. A., eds) 305-319 (Alan Liss, New York; 1988).

CV-N consists of a single chain containing 101 residues and its amino-acid sequence shows obvious duplication. The primary structure of CV-N can be divided into two very similar parts that consist of residues 1-50 and 50-101, respectively. The primary sequence and disulfide bonding pattern were determined by conventional biochemical techniques (Boyd, M. R. et al. Discovery of cyanovirin-N, a novel human immunodeficiency virus-inactivating protein that binds viral surface envelope glycoprotein gp120: potential applications to microbicide development. Antimicrob. Agents Chemother. 41, 1521-1530 (1997); Gustafson, K. R. et al. Isolation, primary sequence determination, and disulfide bond structure of cyanovirin-N, an anti-HIV protein from the cyanobacterium Nostoc ellipsosporum. Biochem. Biophys. Res. Comm. 238, 223-228 (1997)), and a synthetic gene was constructed for over-expression of the protein (Mori, T. et al. Recombinant production of cyanovirin-N, a potent human immunodeficiency virus-inactivating protein derived from a cultured cyanobacterium. Protein Exp. Purific. 12, 151-158 (1998)). Two internal repeats of 50 and 51 amino acids show strong sequence similarity to one another, and equivalent positions of the disulfide bonds (Gustafson, K. R. et al. Isolation, primary sequence determination, and disulfide bond structure of cyanovirin-N, an anti-HIV protein from the cyanobacterium Nostoc ellipsosporum. Biochem. Biophys. Res. Comm. 238, 223-228 (1997)). It has further been shown that cyanovirin-N is extremely resistant to physico-chemical degradation and can withstand treatment with denaturants, detergents, organic solvents such as acetonitrile or methanol, multiple freeze-thaw cycles, and heat (up to 100° C.) with no subsequent loss of antiviral activity (Boyd et al. as above). The primary sequence of cyanovirin-N shares no similarity with other proteins thus far deposited in public protein data bases (Bewley et al. Nature Structural Biology 5, 571-578 (1998)).

In certain embodiments, the term “cyanovirin” (CV-N) as used herein is meant to refer to an isolated or purified protein consisting essentially of SEQ ID NO: 2, as well as antiviral fragments thereof, whether isolated or purified from nature, recombinantly produced, or synthesized, and substantially identical or homologous proteins (as defined herein). An antiviral fragment can be generated, for example, by removing 1-20, preferably 1-10, more preferably 1, 2, 3, 4, or 5, and most preferably 1 or 2, amino acids from one or both ends, preferably from only one end, and most preferably from the amino-terminal end, of the wild-type cyanovirin, such as wild-type cyanovirin of SEQ ID NO: 2. SEQ ID NO: 2 is shown below, and corresponds to NCBI Accession No. P81180.

| SEQ ID NO: 2 | |
|---|---|
| 1 | lgkfsqtcyn saiqgsvlts tcertnggyn tssidlnsvi envdgslkwq psnfietcrn |
| 61 | tqlagssela aecktraqqf vstkinlddh ianidgtlky e |

Interactions between cyanovirin-N and the HIV envelope glycoprotein gp120 have been suggested to account for the antiviral activity of cyanovirin-N (Bewley et al. (1998) as above). Through a variety of experimental approaches, cyanovirin-N was shown to bind avidly to gp120, including re-combinant non-glycosylated gp120. Further, pretreatment of cyanovirin-N with exogenous, virus-free gp120 resulted in a concentration-dependent decrease in antiviral activity4. The recombinant cyanovirin-N used in the NMR structural studies had gp120 binding and anti-HIV properties that were indistinguishable from those of cyanovirin-N isolated from its natural source (Boyd, M. R. et al. Discovery of cyanovirin-N, a novel human immunodeficiency virus-inactivating protein that binds viral surface envelope glycoprotein gp120: potential applications to microbicide development. Antimicrob. Agents Chemother. 41, 1521-1530 (1997)).

Since its identification, methods have been developed for the recombinant production of cyanovirin-N in Escherichia coli (Mori, T. et al., Protein Expr. Purif. 12:151-158, 1998). Cyanovirin-N is an 11 kDa protein consisting of a single 101-amino acid chain containing two intra-chain disulfide bonds. CV-N is an elongated, largely beta-sheet protein that displays internal two fold pseudosymmetry and binds with high affinity and specificity to the HIV surface envelope protein, gp120 (Bewley, C. R. et al., Nature Structural Biology 5(7):571-578, 1998).

Despite its observed anti-viral activity, development of cyanovirin-N protein therapies has been hampered by its relatively short half-life after administration, as well as its in-vivo immunogenicity and potential toxic side effects. Most proteins, particularly relatively low molecular weight proteins introduced into the circulation, are cleared quickly from the mammalian subject by the kidneys. This problem may be partially overcome by administering large amounts of a therapeutic protein or through frequent dosing. However, higher doses of a protein can elicit antibodies that can bind and inactivate the protein and/or facilitate the clearance of the protein from the subject's body. In this way, repeated administration of such therapeutic proteins can essentially become ineffective. Additionally, such an approach may be dangerous since it can elicit an allergic response. Various attempts to solve the problems associated with protein therapies include microencapsulation, liposome delivery systems, administration of fusion proteins, and chemical modification. The most promising of these to date is modification of a therapeutic protein by covalent attachment of poly(alkylene oxide) polymers, particularly polyethylene glycols (“PEG”). For example, Roberts, M. et al., Adv. Drug Delivery Reviews 54 (2002), 459-476, describes the covalent modification of biological macromolecules with PEG to provide physiologically active, non-immunogenic water-soluble PEG conjugates. Methods of attaching PEG to therapeutic molecules, including proteins, are also disclosed in, for example, U.S. Pat. Nos. 4,179,337, 5,122,614, 5,446,090, 5,990,237, 6,214,966, 6,376,604, 6,413,507, 6,495,659, and 6,602,498, each of which is incorporated by herein by reference in its entirety.

### Griffithsin (GRFT)

GRFT was isolated from the red alga Griffithsia sp. collected from the waters off New Zealand. GRFT was shown to display picomolar activity against HIV-1 (Mori et al., 2005), moderately interfering with the binding of gp120 to sCD4. The binding of GRFT to soluble gp120 was inhibited by glucose, mannose, and N-acetylglucosamine (Mori et al., 2005 Isolation and characterization of griffithsin, a novel HIV-inactivating protein, from the red alga Griffithsia sp. J Biol Chem 280: 9345-9353). In addition to inhibiting HIV-1, GRFT was shown to inhibit replication and cytopathy of the coronavirus that causes SARS (Ziólkowska et al., 2006. Domain-swapped structure of the potent antiviral protein griffithsin and its mode of carbohydrate binding. Structure 7: 1127-1135.). The gene encoding GRFT has not been isolated, but the amino-acid sequence was obtained directly from protein purified from cyanobacteria. A GRFT molecule consists of a single 121-amino-acid chain. Analysis of the sequence of GRFT has shown limited homology (less than 30% identity) to proteins such as jacalin (Aucouturier et al., 1987. Characterization of jacalin, the human IgA and IgD binding lectin from jackfruit. Mol Immunol 24:503-511.), heltuba (Bourne et al., 1999. Helianthus tuberosus lectin reveals a widespread scaffold for mannose-binding lectins. Structure Fold Des 7: 1473-1482) or artocarpin (Jeyaprakash et al., 2004. Helianthus tuberosus lectin reveals a widespread scaffold for mannose-binding lectins. Structure Fold Des 7: 1473-1482.), all members of the β-prism-I family of lectins (Raval et al., 2004. A database analysis of jacalin-like lectins: sequence-structure function relationships. Glycobiology 14: 1247-1263; Chandra, 2006. Common scaffolds, diverse recognition profiles. Structure 14: 1093-1094).

GRFT used for biological and structural studies has been prepared as recombinant protein in either E. coli (Giomarelli et al., 2006. Recombinant production of anti-HIV protein, griffithsin, by auto-induction in a fermentor culture. Protein Expr Purif 47:194-202) or Nicothiana benthamiana (Ziólkowska et al., 2006). In both constructs, residue 31 of GRFT was replaced by an alanine, and this substitution did not seem to affect the carbohydrate binding properties of the lectin. GRFT expressed in E. coli contained a N-terminal 6-His affinity tag followed by a putative thrombin cleavage site, extending the protein sequence by 17 amino acids (Mori et al., 2005 Isolation and characterization of griffithsin, a novel HIV-inactivating protein, from the red alga Griffithsia sp. J Biol Chem 280: 9345-9353; Giomarelli et al., 2006); the additional sequence could not be removed and was present in the crystallized protein. The plant-expressed construct did not include any tags, thus resembling more closely the authentic protein, although with an acetylated N terminus and mutated residue 31 (Ziólkowska et al., 2006). Although both the His-tagged and the plant-expressed GRFT crystallized easily, crystals grown from the plant-produced material diffracted significantly better, most likely due to the absence of the extension of the polypeptide chain. Crystals of the His-tagged griffithsin contained only a single molecule in the asymmetric unit (PDB code 2gux) whereas all crystal forms grown from the plant-expressed material contained two molecules (PDB codes 2gty, 2gue, 2guc, 2gud, 2hyr, 2hyq (Ziólkowska et al., 2006; 2007. Crystallographic, thermodynamic, and molecular modeling studies of the mode of binding of oligosaccharides to the potent antiviral protein griffithsin. Proteins: Struct Funct Bioinform.).

The fold of GRFT corresponds to the β-prism-I (Chothia & Murzin, 1993. New folds for all-β proteins. Structure 1: 217-22), observed in a variety of lectins, as well as in some other proteins (Shimizu et al. 1996. The β-prism: a new folding motif. Trends Biochem Sci 21: 3-6). The motif consists of three repeats of anti-parallel four-stranded β-sheet that form a triangular prism. Unlike other members of the family, GRFT forms a domain swapped dimer in which the first two β-strands of one chain are associated with ten strands of the other chain and vice versa (Ziólkowska et al., 2006).

Unlike other proteins that belong to the same fold family, a single molecule of GRFT contains three almost identical carbohydrate-binding sites, each capable of binding a monosaccharide through multiple contact points. The six principal sites in the obligatory dimer of GRFT are very similar and are arranged on every monomer in groups of three. The carbohydrate-binding sites are formed from the parts of the structure that exhibit extensive sequence conservation, but some of the main chain atoms are involved in specific, but sequence-independent contacts with the carbohydrate molecules; these contacts are very similar in all three sites.

GRFT contains three strictly conserved repeats of a sequence GGSGG, located in loops that connect the first and fourth strand of each β-sheet. The main chain amide of the last residue of each of these sequences participates in creation of a ligand-binding site and the strict conservation of this sequence may be the most important reason for the presence of three monosaccharide binding sites on each molecule of GRFT. With one known exception, each molecule of the other lectins that are structurally closely related to GRFT contains only a single carbohydrate binding site. Thus the presence of binding site 1 was reported for all β-prism-I lectins, binding site 2 has only been seen in banana lectin (Meagher et al., 2005. Crystal structure of banana lectin reveals a novel second sugar binding site. Glycobiology 15: 1033-1042.), whereas binding site 3 is unique to GRFT. Three sugar-binding sites of GRFT form an almost perfect equilateral triangle on the edge of the protein, with the carbohydrate molecules found about 15 Å from each other. Very similar interactions are also present in the complexes of GRFT with disaccharides, where the additional sugar units make between zero and two hydrogen bonds with the protein (Ziólkowska et al., 2007).

The reported biological activity of GRFT against HIV is >1 000-fold higher than the activities reported for several monosaccharide-specific lectins (Charan et al., 2000. Isolation and characterization of Myrianthus holstii lectin, a potent HIV-1 inhibitory protein from the plant Myrianthus holstii. J Nat Prod 63: 1170-1174.; Ziólkowska et al., 2006). Since GRFT offers six separate binding sites for mannose in a dimer, the binding potential for the high-mannose oligosaccharides found on the HIV gp120 is significant.

In certain embodiments, the term “griffithsin” (GRFT) as used herein is meant to refer to an isolated or purified protein consisting essentially of SEQ ID NO: 3, as well as antiviral fragments thereof, whether isolated or purified from nature, recombinantly produced, or synthesized, and substantially identical or homologous proteins (as defined herein). An antiviral fragment can be generated, for example, by removing 1-20, preferably 1-10, more preferably 1, 2, 3, 4, or 5, and most preferably 1 or 2, amino acids from one or both ends, preferably from only one end, and most preferably from the amino-terminal end, of the wild-type griffithsin, such as wild-type griffithsin of SEQ ID NO: 3. SEQ ID NO: 3 is shown below, and corresponds to NCBI Accession No. P84801.

| SEQ ID NO: 3 | |
|---|---|
| 1 | slthrkfggs ggspfsglss iavrsgsyld xiiidgvhhg gsggnlsptf tfgsgeyisn |
| 61 | mtirsgdyid nisfetnmgr rfgpyggsgg santlsnvkv iqingsagdy ldsldiyyeq |
| 121 | y |

### Variants

The invention features in certain embodiments variants of CV-N, SVN, GRFT.

The invention features, in certain examples, an isolated or purified antiviral protein comprising the amino acid sequence of SEQ ID NO: 1, an amino acid sequence that is about 90% or more identical to SEQ ID NO: 1, an amino acid sequence that is about 60%, 70%, 75%, 80%, 85%, 90% or more homologous to SEQ ID NO: 1, or a fragment thereof; (ii) an isolated or purified nucleic acid comprising a nucleotide sequence encoding the amino acid sequence of SEQ ID NO: 1; (iii) an isolated or purified antiviral protein comprising the amino acid sequence of SEQ ID NO: 2, an amino acid sequence that is about 90% or more identical to SEQ ID NO: 2, an amino acid sequence that is about 60%, 70%, 75%, 80%, 85%, 90% or more homologous to SEQ ID NO: 2, or a fragment thereof; (iv) an isolated or purified nucleic acid comprising a nucleotide sequence encoding the amino acid sequence of SEQ ID NO: 2; (v) an isolated or purified antiviral protein comprising the amino acid sequence of SEQ ID NO: 3, an amino acid sequence that is about 60%, 70%, 75%, 80%, 85%, 90% or more identical to SEQ ID NO: 3, an amino acid sequence that is about 60%, 70%, 75%, 80%, 85%, 90% or more homologous to SEQ ID NO: 3; (vi) an isolated or purified nucleic acid comprising a nucleotide sequence encoding the amino acid sequence of SEQ ID NO: 3, or a fragment thereof.

The term “homology” as used herein is meant to refer to the percent identity between two polynucleotide or two polypeptide moieties. Two DNA, or two polypeptide sequences are “substantially homologous” to each other when the sequences exhibit at least about 50%, preferably at least about 75%, more preferably at least about 80%-85%, preferably at least about 90%, and most preferably at least about 95%-98%, or more, sequence identity over a defined length of the molecules. As used herein, substantially homologous also refers to sequences showing complete identity to the specified DNA or polypeptide sequence.

The term “identity” or “identical” as used herein refers to an exact nucleotide-to-nucleotide or amino acid-to-amino acid correspondence of two polynucleotides or polypeptide sequences, respectively.

When the above isolated or purified nucleic acid is characterized in terms of “percentage of sequence identity,” a given nucleic acid molecule as described above is compared to a nucleic acid molecule encoding a corresponding gene (i.e., the reference sequence) by optimally aligning the nucleic acid sequences over a comparison window, wherein the portion of the polynucleotide sequence in the comparison window may comprise additions or deletions (i.e., gaps) as compared to the reference sequence, which does not comprise additions or deletions, for optimal alignment of the two sequences. The percentage of sequence identity is calculated by determining the number of positions at which the identical nucleic acid base occurs in both sequences, i.e., the number of matched positions, dividing the number of matched positions by the total number of positions in the window of comparison, and multiplying the result by 100 to yield the percentage of sequence identity. Optimal alignment of sequences for comparison may be conducted by computerized implementations of known algorithms (e.g., GAP, BESTFIT, FASTA, and TFASTA in the Wisconsin Genetics Software Package, Genetics Computer Group (GCG), 575 Science Dr., Madison, Wis., or BlastN and BlastX available from the National Center for Biotechnology Information, Bethesda, Md.), or by inspection. Sequences are typically compared using BESTFIT or BlastN with default parameters.

“Substantial sequence identity” means that about 60%, preferably about 65%, more preferably about 70%, still more preferably about 75%, even more preferably about 80%, even still more preferably about 85%, and most preferably about 90% or more of the sequence of a given nucleic acid molecule is identical to a given reference sequence. Typically, two polypeptides are considered to be substantially identical if about 60%, preferably about 65%, more preferably about 70%, still more preferably about 75%, even more preferably about 80%, even still more preferably about 85%, and most preferably about 90% or more of the amino acids of which the polypeptides are comprised are identical to or represent conservative substitutions of the amino acids of a given reference sequence.

Another indication that polynucleotide sequences are substantially identical is if two molecules selectively hybridize to each other under stringent conditions. The phrase “selectively hybridizing to” refers to the selective binding of a single-stranded nucleic acid probe to a single-stranded target DNA or RNA sequence of complementary sequence when the target sequence is present in a preparation of heterogeneous DNA and/or RNA. Stringent conditions are sequence-dependent and will be different in different circumstances. Generally, stringent conditions are selected to be about 2 C. lower than the thermal melting point (Tm) for the specific sequence at a defined ionic strength and pH. The Tm is the temperature (under defined ionic strength and pH) at which 50% of the target sequence hybridizes to a perfectly matched probe.

In view of the above, “stringent conditions” preferably allow up to about 25% mismatch, more preferably up to about 15% mismatch, and most preferably up to about 10% mismatch. “At least moderately stringent conditions” preferably allow for up to about 40% mismatch, more preferably up to about 30% mismatch, and most preferably up to about 20% mismatch. “Low stringency conditions” preferably allow for up to about 60% mismatch, more preferably up to about 50% mismatch, and most preferably up to about 40% mismatch. Hybridization and wash conditions that result in such levels of stringency can be selected by the ordinarily skilled artisan using the references cited under “EXAMPLES” among others.

One of ordinary skill in the art will appreciate, however, that two polynucleotide sequences can be substantially different at the nucleic acid level, yet encode substantially similar, if not identical, amino acid sequences, due to the degeneracy of the genetic code. The present invention is intended to encompass such polynucleotide sequences.

A variety of techniques used to synthesize the oligonucleotides of the present invention are known in the art. See, for example, Lemaitre et al., PNAS USA 84: 648-652 (1987).

Given the present disclosure, it will be apparent to one ordinarily skilled in the art that certain modified scytovirin gene sequences will code for a fully functional, i.e., antiviral, such as anti-HCV, scytovirin homolog. A minimum essential DNA coding sequence(s) for a functional scytovirin can readily be determined by one skilled in the art, for example, by synthesis and evaluation of sub-sequences comprising the wild-type scytovirin, and by site-directed mutagenesis studies of the scytovirin DNA coding sequence.

In certain examples, the variant comprises one or more conservative or neutral amino acid substitutions or one or more amino acid additions at the N-terminus or C-terminus, wherein the variant has antiviral activity characteristic of the antiviral protein consisting essentially of the amino acid sequence of SEQ ID NO: 1, SEQ ID NO: 2 or SEQ ID NO: 3.

Variants may, in certain examples, comprise CV-N, SVN, GRFT polypeptides with one or more amino acid substitutions.

### Amino Acid Substitutions

It is well known in the art that one or more amino acids in a native sequence can be substituted with other amino acid(s) having similar charge and polarity, i.e., a conservative amino acid substitution, resulting in a silent change. Conservative substitutions for an amino acid within the native polypeptide sequence can be selected from other members of the class to which the amino acid belongs.

The 20 amino acids found in naturally occurring proteins can be generally classified as polar (S, T, C, Y, D, N, E, Q, R, H, K) or non-polar (G, A, V, L, I, M, F, W, P). They can be further classified into four major classes; namely, acidic, basic, neutral/polar and neutral/nonpolar, where the first three classes fall under the general heading of “polar” above. These four classes have the following characteristics:

Acidic: A significant percentage (e.g. at least 25%) of molecules are negatively charged (due to loss of H+ion) in aqueous solution at physiological pH.

Basic: A significant percentage (e.g. at least 25%) of molecules are positively charged (due to association with H+ion) in aqueous solution at physiological pH.

Both acidic and basic residues are attracted by aqueous solution, so as to seek outer surface positions in the conformation of a peptide in aqueous medium at physiological pH.

Neutral/polar: The residues are uncharged at physiological pH but are also attracted by aqueous solution, so as to seek outer surface positions in the conformation of a peptide in aqueous medium.

Neutral/non-polar: The residues are uncharged at physiological pH and are repelled by aqueous solution, so as to seek internal positions in the conformation of a peptide in aqueous medium. These residues are also designated “hydrophobic”.

Amino acid residues can be further subclassified as cyclic/noncyclic and aromatic/nonaromatic, with respect to the side chain substituent groups of the residues, and as small or large. The residue is considered small if it contains a total of 4 carbon atoms or less, inclusive of the carboxyl carbon.

Subclassification of the naturally occurring protein amino acids according to the foregoing scheme is as follows:

Acidic: Aspartic acid and Glutamic acid

Basic/noncyclic: Arginine and Lysine

Basic/cyclic: Histidine

Neutral/polar/small: Threonine, Serine and Cysteine

Neutral/polar/large/nonaromatic: Asparagine and Glutamine

Neutral/polar/large/aromatic: Tyrosine

Neutral/non-polar/small: Alanine
Neutral/non-polar/large/nonaromatic: Valine, Isoleucine, Leucine, and

Methionine

Neutral/non-polar/large/aromatic: Phenylalanine and Tryptophan

Proline, technically falling within the group neutral/non-polar/large/cyclic and nonaromatic, is considered a special case due to its known effects on the secondary conformation of peptide chains, and is not, therefore, included in this defined group, but is regarded as a group of its own.

The role of the hydropathic index of amino acids in conferring interactive biological function on a protein may be considered. See, for example, Kyte and Doolittle, J. Mol. Biol. 157:105-132 (1982). It is accepted that the relative hydropathic character of amino acids contributes to the secondary structure of the resultant protein, which in turn defines the interaction of the protein with other molecules, e.g., enzymes, substrates, receptors, DNA, antibodies, antigens, etc. It is also understood in the art that the substitution of like amino acids may be made effectively on the basis of hydrophilicity, as the greatest local average hydrophilicity of a protein is known to correlate with a biological property of the protein. See, for example, U.S. Pat. No. 4,554,101, incorporated by reference in its entirety herein. Each amino acid has been assigned a hydropathic index and a hydrophilic value, listed as follows: Alanine +1.8−0.5 Cysteine +2.5−1.0 Aspartic acid −3.5+3.0.+−.1 Glutamic acid −3.5+3.0.+−.1 Phenylalanine +2.8−2.5 Glycine −0.4 0 Histidine −3.2−0.5 Isoleucine +4.5−1.8 Lysine −3.9+3.0 Leucine +3.8−1.8 Methionine +1.9−1.3 Asparagine −3.5+0.2 Proline −1.6−0.5.+−.1 Glutamine −3.5+0.2 Arginine −4.5+3.0 Serine −0.8+0.3 Threonine −0.7−0.4 Valine +4.2−1.5 Tryptophan −0.9−3.4 Tyrosine −1.3−2.3

It is known in the art that certain amino acids may be substituted by other amino acid having a similar hydropathic or hydrophilic index, score or value, and result in a protein with similar biological activity. The substitution of amino acids whose hydropathic indices or hydrophilic values are within .+−.2 is preferred, those within .+−.1 are more preferred, and those within .+−.0.5 are most preferred.

As outlined above, conservative amino acid substitutions are therefore based on the relative similarity of the amino acid side-chain substituents, for example, their hydrophobicity, hydrophilicity, charge, size, and the like. Exemplary substitutions which take various of the foregoing characteristics into consideration are well known to those of skill in the art and include: arginine/lysine; glutamate/aspartate; serine/threonine; glutamine/asparagine; and valine/leucine/isoleucine.

The CV-N variants of the invention may also include commonly encountered amino acids which do not occur naturally in proteins, such as .beta.-alanine, other omega-amino acids, such as 4-amino butyric acid, and so forth; a-aminoisobutyric acid (Aib), sarcosine (Sar), ornithine (Om), citrulline (Cit), t-butylalanine (t-BuA), t-butylglycine (t-BuG), N-methylisoleucine (N-Melle), phenylglycine (Phg), cyclohexylalanine (Cha), norleucine (Nle), cysteic acid (Cya), and methionine sulfoxide (MSO). These amino acids can also be classifed by the above scheme, as follows: Sar and .beta.-Ala are neutral/non-polar/small; t-BuA, t-BuG, N-Melle, Nle and Cha are neutral/non-polar/large/nonaromatic; Om is basic/noncyclic; Cya is acidic; Cit, Acetyl Lys, and MSO are neutral/polar/large/nonaromatic; and Phg is neutral/non-polar/large/aromatic.

The various omega-amino acids are classified according to size as neutral/non-polar/small (.beta.-Ala, 4-aminobutyric) or large (all others). Accordingly, conservative substitutions using these amino acids can be determined.

In a preferred aspect of the invention, biologically functional equivalents of the polypeptides or fragments thereof have about 25 or fewer conservative amino acid substitutions, more preferably about 15 or fewer conservative amino acid substitutions, and most preferably about 10 or fewer conservative amino acid substitutions. In further preferred embodiments, the polypeptide has between 1 and 10, between 1 and 7, or between 1 and 5 conservative substitutions. In selected embodiments, the polypeptide has 1, 2, 3, 4, or 5 conservative amino acid substitutions. In each case, the substitution(s) are preferably at the preferred amino acid residues of native CV-N noted below.

Non-conservative substitutions include additions, deletions, and substitutions that do not fall within the criteria given above for conservative substitutions. Non-conservative substitutions are preferably limited to regions of the protein which are remote, in a three-dimensional sense, from the mannose-binding sites that permit binding of CV-N to gp120 and other high mannose proteins (see below). Preferably, the protein has 15 or fewer non-conservative amino acid substitutions, more preferably 10 or fewer non-conservative amino acid substitutions. In further preferred embodiments, the polypeptide has fewer than 5 non-conservative substitutions. In selected embodiments, the polypeptide has 0, 1, 2, or 3 non-conservative amino acid substitutions.

### Viral Vectors and Transformation

Viral vectors are a kind of expression construct that utilize viral sequences to introduce nucleic acid and possibly proteins into a cell. The ability of certain viruses to infect cells or enter cells via receptor-mediated endocytosis, and to integrate into host cell genome and express viral genes stably and efficiently have made them attractive candidates for the transfer of foreign nucleic acids into cells (e.g., mammalian cells). Vector components of the present invention may be a viral vector that encode one or more candidate substance or other components such as, for example, an immunomodulator or adjuvant for the candidate substance. Non-limiting examples of virus vectors that may be used to deliver a nucleic acid of the present invention are described herein.

One method for delivery of the nucleic acid involves the use of an adenovirus expression vector. “Adenovirus expression vector” is meant to include those constructs containing adenovirus sequences sufficient to (a) support packaging of the construct and (b) to ultimately express a tissue or cell-specific construct that has been cloned therein. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992).

The nucleic acid may be introduced into the cell using adenovirus assisted transfection. Increased transfection efficiencies have been reported in cell systems using adenovirus coupled systems (Kelleher and Vos, 1994; Cotten et al., 1992; Curiel, 1994). Adeno-associated virus (AAV) is an attractive vector system for use in the candidate substances of the present invention as it has a high frequency of integration and it can infect non-dividing cells, thus making it useful for delivery of genes into mammalian cells, for example, in tissue culture (Muzyczka, 1992) or in vivo. Details concerning the generation and use of rAAV vectors are described in U.S. Pat. Nos. 5,139,941 and 4,797,368, each incorporated herein by reference.

Retroviruses may be used. In order to construct a retroviral vector, a nucleic acid (e.g., one encoding a single chain antibody described herein) is inserted into the viral genome in the place of certain viral sequences to produce a virus that is replication-defective. In order to produce virions, a packaging cell line containing the gag, pol, and env genes but without the LTR and packaging components is constructed Mann et al., 1983).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell is described in U.S. Pat. No. 5,994,136, incorporated herein by reference.

Other viral vectors that may be used include vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

### Delivery

Suitable methods for nucleic acid delivery for transformation of a cell, a tissue or an organism for use with the current invention are believed to include virtually any method by which a nucleic acid (e.g., DNA) can be introduced into a cell, a tissue or an organism, as described herein or as would be known to one of ordinary skill in the art. Such methods include, but are not limited to, direct delivery of DNA such as by ex vivo transfection (Wilson et al., 1989, Nabel et al., 1989), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harlan and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (WO 94/09699 and WO 95/06128; U.S. Pat. Nos. 5,610,042, 5,322,783, 5,563,055, 5,550,318, 5,538,877 and 5,538,880, each of which is incorporated herein by reference); by agitation with silicon carbide fibers (Kaeppler et al., 1990; U.S. Pat. Nos. 5,302,523 and 5,464,765, each incorporated herein by reference); by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985), and any combination of such methods. Through the application of techniques such as these, cell(s), tissue(s) or organism(s) may be stably or transiently transformed.

### Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny. As used herein, the terms “engineered” and “recombinant” cells or host cells are intended to refer to a cell into which an exogenous nucleic acid sequence, such as, for example, a vector, has been introduced. Therefore, recombinant cells are distinguishable from naturally occurring cells which do not contain a recombinantly introduced nucleic acid.

It is also contemplated that RNAs or proteinaceous sequences may be co-expressed with other selected RNAs or proteinaceous sequences in the same host cell. Co-expression may be achieved by co-transfecting the host cell with two or more distinct recombinant vectors. Alternatively, a single recombinant vector may be constructed to include multiple distinct coding regions for RNAs, which could then be expressed in host cells transfected with the single vector.

In certain embodiments, the host cell or tissue may be comprised in at least one organism. In certain embodiments, the organism may be, but is not limited to, a prokaryote (e.g., a eubacteria, an archaea) or an eukaryote, as would be understood by one of ordinary skill in the art (see, for example, webpage phylogeny.arizona.edu/tree/phylogeny.html).

Numerous cell lines and cultures are available for use as a host cell, and they can be obtained through the American Type Culture Collection (ATCC), which is an organization that serves as an archive for living cultures and genetic materials (www.atcc.org) or through various vendors and commercial sources that cell expression systems. An appropriate host can be determined by one of skill in the art based on the vector backbone and the desired result. A plasmid or cosmid, for example, can be introduced into a prokaryote host cell for replication of many vectors. Cell types available for vector replication and/or expression include, but are not limited to, bacteria, such as E. coli (e.g., E. coli strain RR1, E. coli LE392, E. coli B, E. coli X 1776 (ATCC No. 31537) as well as E. coli W3110 (F-, lambda-, prototrophic, ATCC No. 273325), DH5-alpha, JM109, and KC8, bacilli such as Bacillus subtilis; and other enterobacteriaceae such as Salmonella typhimurium, Serratia marcescens, various Pseudomonas species, as well as a number of commercially available bacterial hosts.

Examples of eukaryotic host cells for replication and/or expression of a vector include, but are not limited to, HeLa, NIH3T3, Jurkat, 293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

It is an aspect of the present invention that the nucleic acid compositions described herein may be used in conjunction with a host cell. For example, a host cell may be transfected using all or part of SEQ ID NO: 1, 2 or 3, a fragment, variant or a similar sequences.

### Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

For example, the insect cell/baculovirus system can produce a high level of protein expression of a heterologous nucleic acid segment, such as described in U.S. Pat. Nos. 5,871,986, 4,879,236, both herein incorporated by reference, and which are commercially available.

Other examples of expression systems include Inducible Mammalian Expression Systems (e.g. commercially available from STRATAGENE), which involves a synthetic ecdysone-inducible receptor, or its pET Expression System, an E. coli expression system. Another example of an inducible expression system is available from INVITROGEN, which carries tetracycline-regulated expression that uses the full-length CMV promoter. One of skill in the art would know how to express a vector, such as an expression construct, to produce a nucleic acid sequence or its cognate polypeptide, protein, or peptide.

It is contemplated that the proteins, polypeptides or peptides produced by the methods of the invention may be “overexpressed,” i.e., expressed in increased levels relative to its natural expression in cells. Such overexpression may be assessed by a variety of methods, including radio-labeling and/or protein purification. However, simple and direct methods are preferred, for example, those involving SDS/PAGE and protein staining or western blotting, followed by quantitative analyses, such as densitometric scanning of the resultant gel or blot.

### Antibodies

Also provided are anti-scytovirin, anti-cyanovirin or anti-griffithsin antibodies for use in the methods as claimed.

The term “epitope” as used herein refers to a sequence of at least about 3 to 5, preferably about 5 to 10 or 15, and not more than about 1,000 amino acids (or any integer value between 3 and 1,000), which define a sequence that by itself or as part of a larger sequence, binds to an antibody generated in response to such sequence. There is no critical upper limit to the length of the fragment, which may comprise nearly the full-length of the protein sequence, or even a fusion protein comprising two or more epitopes. An epitope for use in the subject invention is not limited to a polypeptide having the exact sequence of the portion of the parent protein from which it is derived. Indeed, viral genomes are in a state of constant flux and contain several variable domains which exhibit relatively high degrees of variability between isolates. Thus the term “epitope” encompasses sequences identical to the native sequence, as well as modifications to the native sequence, such as deletions, additions and substitutions (generally conservative in nature).

Regions of a given polypeptide that include an epitope can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N.J. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1986) Molec. Immunol. 23:709-715, all incorporated herein by reference in their entireties. Similarly, conformational epitopes are readily identified by determining spatial conformation of amino acids such as by, e.g., x-ray crystallography and 2-dimensional nuclear magnetic resonance. See, e.g., Epitope Mapping Protocols, supra. Antigenic regions of proteins can also be identified using standard antigenicity and hydropathy plots, such as those calculated using, e.g., the Omiga version 1.0 software program available from the Oxford Molecular Group. This computer program employs the Hopp/Woods method, Hopp et al., Proc. Natl. Acad. Sci. USA (1981) 78:3824-3828 for determining antigenicity profiles, and the Kyte-Doolittle technique, Kyte et al., J. Mol. Biol. (1982) 157:105-132 for hydropathy plots.

In certain examples, matrix-anchored anti-scytovirin, anti-cyanovirin or anti-griffithsin antibodies can be used in a method to inhibit virus in a sample. Preferably, the antibody binds to an epitope consisting essentially of SEQ ID NO: 1, SEQ ID NO: 2 or SEQ ID NO: 3. The antibody can be coupled to a solid support matrix using similar methods and with similar considerations as described above for attaching a scytovirin to a solid support matrix. In one example, coupling methods and molecules employed to attach an anti-scytovirin antibody to a solid support matrix, such as magnetic beads or a flow-through matrix, can employ biotin/streptavidin coupling or coupling through molecules, such as polyethylene glycol, albumin or dextran. Also analogously, it can be shown that, after such coupling, the matrix-anchored anti-scytovirin antibody retains its ability to bind to a scytovirin consisting essentially of SEQ ID NO: 1, which protein can inhibit a virus. Preferably, the matrix is a solid support matrix, such as a magnetic bead or a flow-through matrix. If the solid support matrix to which the anti-scytovirin antibody is attached comprises magnetic beads, removal of the antibody-scytovirin complex can be readily accomplished using a magnet.

Antibodies as described herein are of use in the methods of the invention. For example, the antibodies can be used in a method of treating or preventing a viral infection in a subject comprising administering to the subject one or more antibodies selected from: (i) an antibody that binds a protein comprising the amino acid sequence of SEQ ID NO: 1; (ii) an antibody that binds a protein comprising the amino acid sequence of SEQ ID NO: 2; (iii) an antibody that binds a protein comprising the amino acid sequence of SEQ ID NO: 3, or a fragment thereof, in an amount sufficient to induce in the subject an immune response to the virus; thereby treating or preventing the viral infection in a subject.

In certain examples, the viral infection is caused by a virus with a coat protein comprising high-mannose oligosaccharides. For instance, the virus in certain embodiments is hepatitis C virus (HCV). In other certain examples, the virus is human immunodeficiency virus (HIV).

Thus, a scytovirin, a cyanovirin, or a griffithsin can be administered to an animal, the animal generates the corresponding antibodies (e.g. administered scytovirin and generates anti-scytovirin antibodies). Certain of the antibodies have an internal image that recognizes the target site in the HCV or HIV, e.g. the targeting epitope. In accordance with well-known methods, polyclonal or monoclonal antibodies can be obtained, isolated and selected. Such antibodies can be administered to an animal to inhibit a viral infection in accordance with methods provided herein.

Although nonhuman anti-idiotypic antibodies are proving useful as vaccine antigens in humans, their favorable properties might, in certain instances, be further enhanced and/or their adverse properties further diminished, through “humanization” strategies, such as those recently reviewed by Vaughan, (Nature Biotech. 16: 535-539 (1998)). Alternatively, a scytovirin or a cyanovirin or a griffithsin can be directly administered to an animal to inhibit a viral infection in accordance with methods provided herein such that the treated animal, itself, generates the corresponding antibody, for example an anti-scytovirin antibody.

Also featured in the invention are methods for elimination of a virus from the blood of a subject, methods of inhibiting a virus in a biological sample, methods of treating or preventing a viral infection caused by a virus in or on the skin or mucous membrane, and methods of inhibiting a virus in or on an object. All of the above-described methods comprise administering to the subject one or more antibodies selected from: (i) an antibody that binds a protein comprising the amino acid sequence of SEQ ID NO: 1; (ii) an antibody that binds a protein comprising the amino acid sequence of SEQ ID NO: 2; (iii) an antibody that binds a protein comprising the amino acid sequence of SEQ ID NO: 3, or a fragment thereof, in an amount sufficient to induce in the subject an immune response to the virus; and thereby inhibiting the virus in a biological sample.

The methods of the invention can further comprise the administration of one or more additional agents, for example, but not limited to additional therapeutic agents or immunostimulants.

With respect to the above methods, sufficient amounts can be determined in accordance with methods known in the art. Similarly, the sufficiency of an immune response in the inhibition of a viral infection in an animal also can be assessed in accordance with methods known in the art.

Any of the above methods can further comprise concurrent, pre- or post-treatment with an adjuvant to enhance the immune response, such as the prior, simultaneous or subsequent administration, by the same or a different route, of an antiviral agent or another agent that is efficacious in inducing an immune response to the virus, such as an immunostimulant. See, for example, Harlow et al., 1988, supra.

### Methods

The inventors of the instant application have developed novel compositions and methods for treating and preventing viral infection, and in particular infection by high mannose enveloped viruses.

High mannose enveloped viruses are viruses that viruses that bear high-mannose structures on their surface glycoproteins. “High mannose” is meant to refer to at least six, typically six to nine, linked mannose rings. Any virus that has high mannose glycans present on the viral glycoprotein is considered for use in the invention as described herein. High mannose envelope viruses are meant to include, but are not limited to HCV, HIV, influenza virus, measles virus, herpes virus 6, marburg virus, and ebola virus. In particular embodiments, the virus with a coat protein comprising high-mannose oligosaccharides is selected from, but not limited to, HCV or HIV.

Enabled by the present invention are methods of treating or preventing a viral infection in a subject using compositions comprising SEQ ID NO: 1, SEQ ID NO: 2 or SEQ ID NO: 3, or a combination thereof (e.g. SEQ ID NO: 1 and 2, SEQ ID NO: 1 and 3, SEQ ID NO: 2 and 3, SEQ ID NO: 1, 2 and 3).

Also enabled by the present invention are methods of inhibiting a virus in a biological sample using compositions comprising SEQ ID NO: 1, SEQ ID NO: 2 or SEQ ID NO: 3, or a combination thereof (e.g. SEQ ID NO: 1 and 2, SEQ ID NO: 1 and 3, SEQ ID NO: 2 and 3, SEQ ID NO: 1, 2 and 3).

Also enabled by the present invention are methods of inhibiting a virus in or on an object using compositions comprising SEQ ID NO: 1, SEQ ID NO: 2 or SEQ ID NO: 3, or a combination thereof (e.g. SEQ ID NO: 1 and 2, SEQ ID NO: 1 and 3, SEQ ID NO: 2 and 3, SEQ ID NO: 1, 2 and 3).

The methods, in certain examples, comprise administering to the subject an effective amount of at least one of the following: (i) an isolated or purified antiviral protein comprising the amino acid sequence of SEQ ID NO: 1, an amino acid sequence that is about 90% or more identical to SEQ ID NO: 1, an amino acid sequence that is about 90% or more homologous to SEQ ID NO: 1, or a fragment thereof; (ii) an isolated or purified nucleic acid comprising a nucleotide sequence encoding the amino acid sequence of SEQ ID NO: 1; (iii) an isolated or purified antiviral protein comprising the amino acid sequence of SEQ ID NO: 2, an amino acid sequence that is about 90% or more identical to SEQ ID NO: 2, an amino acid sequence that is about 90% or more homologous to SEQ ID NO: 2, or a fragment thereof; (iv) an isolated or purified nucleic acid comprising a nucleotide sequence encoding the amino acid sequence of SEQ ID NO: 2; (v) an isolated or purified antiviral protein comprising the amino acid sequence of SEQ ID NO: 3, an amino acid sequence that is about 90% or more identical to SEQ ID NO: 3, an amino acid sequence that is about 90% or more homologous to SEQ ID NO: 3; (vi) an isolated or purified nucleic acid comprising a nucleotide sequence encoding the amino acid sequence of SEQ ID NO: 3, or a fragment thereof, and thereby treating or preventing the viral infection in a subject.

Certain methods of the invention may include steps concerning determining or identifying that a subject has been exposed to a sexually transmitted microbe or determining that a subject is a risk for an infection by a sexually transmitted microbe. Thus, steps for assaying for infection or for taking a patient history are included in embodiments of the invention.

The invention features, in certain embodiments, methods of treating or preventing a viral infection caused by a virus in or on the skin or mucous membrane comprising contacting the affected area with a topical composition comprising an effective amount of at least one of the following: (i) an isolated or purified antiviral protein comprising the amino acid sequence of SEQ ID NO: 1, an amino acid sequence that is about 90% or more identical to SEQ ID NO: 1, an amino acid sequence that is about 90% or more homologous to SEQ ID NO: 1, or a fragment thereof; (ii) an isolated or purified nucleic acid comprising a nucleotide sequence encoding the amino acid sequence of SEQ ID NO: 1; (iii) an isolated or purified antiviral protein comprising the amino acid sequence of SEQ ID NO: 2, an amino acid sequence that is about 90% or more identical to SEQ ID NO: 2, an amino acid sequence that is about 90% or more homologous to SEQ ID NO: 2, or a fragment thereof; (iv) an isolated or purified nucleic acid comprising a nucleotide sequence encoding the amino acid sequence of SEQ ID NO: 2; (v) an isolated or purified antiviral protein comprising the amino acid sequence of SEQ ID NO: 3, an amino acid sequence that is about 90% or more identical to SEQ ID NO: 3, an amino acid sequence that is about 90% or more homologous to SEQ ID NO: 3; (vi) an isolated or purified nucleic acid comprising a nucleotide sequence encoding the amino acid sequence of SEQ ID NO: 3, or a fragment thereof, and thereby treating or preventing a viral infection caused by a virus in or on the skin or mucous membrane.

The biological sample can be selected from, but not limited to, blood, a blood product, cells, a tissue, an organ, sperm, a vaccine formulation, and a bodily fluid.

### Hepatitis C

The hepatitis C virus (HCV) is one of the most important causes of chronic liver disease in the United States. It accounts for about 15 percent of acute viral hepatitis, 60 to 70 percent of chronic hepatitis, and up to 50 percent of cirrhosis, end-stage liver disease, and liver cancer. Of the U.S. population, 1.6 percent, or an estimated 4.1 million Americans, have antibody to HCV (anti-HCV), indicating ongoing or previous infection with the virus. Hepatitis C causes an estimated 10,000 to 12,000 deaths annually in the United States.

HCV is one of six known hepatitis viruses: A, B, C, D, E, G. The discovery of HCV was published in 1989 (Isolation of a cDNA clone derived from a blood-borne non-A, non-B viral hepatitis genome; Choo et al., Science 244 (4902): 359-62. 1989). The Hepatitis C virus (HCV) is a small (50 nm in size), enveloped, single-stranded, positive sense RNA virus. It is the only known member of the HCV genus in the family Flaviviridae. There are six major genotypes of the hepatitis C virus, which are indicated numerically (e.g., genotype 1, genotype 2, etc.).

Information on HCV is publicly available on the world wide web at digestive.niddk.nih.gov/ddiseases/pubs/chronichepc/.

A characteristic of hepatitis C is its tendency to cause chronic liver disease in which the liver injury persists for a prolonged period, if not for life. About 75 percent of patients with acute hepatitis C ultimately develop chronic infection.

Chronic hepatitis C varies in its course and outcome. At one end of the spectrum are infected persons who have no signs or symptoms of liver disease and have completely normal levels of serum enzymes, the usual blood test results that indicate liver disease. Liver biopsy usually shows some degree of injury to the liver, but the extent is usually mild, and the overall prognosis may be good. At the other end of the spectrum are patients with severe hepatitis C who have symptoms, high levels of the virus (HCV RNA) in serum, and elevated serum enzymes, and who ultimately develop cirrhosis and end-stage liver disease. In the middle of the spectrum are many patients who have few or no symptoms, mild to moderate elevations in liver enzymes, and an uncertain prognosis.

Chronic hepatitis C can cause cirrhosis, liver failure, and liver cancer. Researchers estimate that at least 20 percent of patients with chronic hepatitis C develop cirrhosis, a process that takes at least 10 to 20 years. Liver failure from chronic hepatitis C is one of the most common reasons for liver transplants in the United States. After 20 to 40 years, a small percentage of patients develop liver cancer. Hepatitis C is the cause of about half of cases of primary liver cancer in the developed world. Men, alcoholics, patients with cirrhosis, people over age 40, and those infected for 20 to 40 years are at higher risk of developing HCV-related liver cancer.

HCV is spread primarily by contact with infected blood and blood products. Blood transfusions and the use of shared, unsterilized, or poorly sterilized needles, syringes and injection equipment or paraphernalia have been the main routes of the spread of HCV in the United States. HCV can be transmitted sexually, and is more likely to occur when an STD (like HIV) is also present and makes blood contact more likely

Assessing or determining if a patient or subject is at risk of HCV infection may entail the assessment of various risk factors. Several activities and practices have been identified as potential sources of exposure to the HCV.

Those who currently use or have used drug injection as their delivery route for illicit drugs are at increased risk for getting hepatitis C because they may be sharing needles or other drug paraphernalia (includes cookers, cotton, spoons, water, etc.), which may be contaminated with HCV-infected blood. It is estimated that 60% to 80% of all IV drug users in the United States have been infected with HCV.

The transmission of HCV may be possible through the nasal inhalation of illegal drugs such as cocaine and crystal methamphetamine when straws (containing even trace amounts of mucus and blood) are shared among users.

HCV was first isolated in 1989 and reliable tests to screen for the virus were not available until 1992. Therefore, those who received blood or blood products prior to the implementation of screening the blood supply for HCV may have been exposed to the virus. Blood products include clotting factors (taken by hemophiliacs), immunoglobulin, platelets, and plasma. In 2001, the Centers for Disease Control and Prevention reported that the risk of HCV infection from a unit of transfused blood in the United States is less than one per million transfused units.

Medical and dental personnel, first responders (e.g., firefighters, paramedics, emergency medical technicians, law enforcement officers), and military combat personnel can be exposed to HCV through accidental exposure to blood through accidental needlesticks or blood spatter to the eyes or open wounds. Universal precautions to protect against such accidental exposures significantly reduce the risk of exposure to HCV.

Personal care items such as razors, toothbrushes, cuticle scissors, and other manicuring or pedicuring equipment can easily be contaminated with blood. Sharing such items can potentially lead to exposure to HCV.

Sporadic transmission, when the source of infection is unknown, is the basis for about 10 percent of acute hepatitis C cases and for 30 percent of chronic hepatitis C cases. These cases are usually referred to as sporadic or community-acquired infections. These infections may have come from exposure to the virus from cuts, wounds, or medical injections or procedures.

Many people with chronic hepatitis C have no symptoms of liver disease. If symptoms are present, they are usually mild, nonspecific, and intermittent. They may include fatigue, mild right-upper-quadrant discomfort or tenderness (“liver pain”), nausea, poor appetite, muscle and joint pains. Similarly, the physical exam is likely to be normal or show only mild enlargement of the liver or tenderness. Some patients have vascular spiders or palmar erythema.

Once a patient develops cirrhosis or if the patient has severe disease, symptoms and signs are more prominent. In addition to fatigue, the patient may complain of muscle weakness, poor appetite, nausea, weight loss, itching, dark urine, fluid retention, and abdominal swelling. Physical findings of cirrhosis may include enlarged liver enlarged spleen, jaundice, muscle wasting, excoriations (scratches or abrasions on the skin), ascites (fluid-filled belly), ankle swelling.

Hepatitis C is most readily diagnosed when serum aminotransferases are elevated and anti-HCV is present in serum. The diagnosis is confirmed by the finding of HCV RNA in serum.

Chronic hepatitis C is diagnosed when anti-HCV is present and serum aminotransferase levels remain elevated for more than 6 months. Testing for HCV RNA (by PCR) confirms the diagnosis and documents that viremia is present; almost all patients with chronic infection will have the viral genome detectable in serum by PCR.

Diagnosis is problematic in patients who cannot produce anti-HCV because they are immunosuppressed or immunoincompetent. Thus, HCV RNA testing may be required for patients who have a solid-organ transplant, are on dialysis, are taking corticosteroids, or have agammaglobulinemia. Diagnosis is also difficult in patients with anti-HCV who have another form of liver disease that might be responsible for the liver injury, such as alcoholism, iron overload, or autoimmunity. In these situations, the anti-HCV may represent a false-positive reaction, previous HCV infection, or mild hepatitis C occurring on top of another liver condition. HCV RNA testing in these situations helps confirm that hepatitis C is contributing to the liver problem.

The therapy for chronic hepatitis C has evolved steadily since alpha interferon was first approved for use in HVC more than 10 years ago. At the present time, the optimal regimen appears to be a 24- or 48-week course of the combination of pegylated alpha interferon and ribavirin.

Alpha interferon is a host protein that is made in response to viral infections and has natural antiviral activity. Recombinant forms of alpha interferon have been produced, and several formulations (alfa-2a, alfa-2b, consensus interferon) are available as therapy for hepatitis C. These standard forms of interferon, however, are now being replaced by pegylated interferon (peginterferon).

Peginterferon is alpha interferon that has been modified chemically by the addition of a large inert molecule of polyethylene glycol. Pegylation changes the uptake, distribution, and excretion of interferon, prolonging its half-life. Peginterferon can be given once weekly and provides a constant level of interferon in the blood, whereas standard interferon must be given several times weekly and provides intermittent and fluctuating levels. In addition, peginterferon is more active than standard interferon in inhibiting HCV and yields higher sustained response rates with similar side effects. Because of its ease of administration and better efficacy, peginterferon has replaced standard interferon both as monotherapy and as combination therapy for hepatitis C.

Ribavirin is an oral antiviral agent that has activity against a broad range of viruses. By itself, ribavirin has little effect on HCV, but adding it to interferon increases the sustained response rate by two- to three-fold. For these reasons, combination therapy is now recommended for hepatitis C, and interferon monotherapy is applied only when there are specific reasons not to use ribavirin.

It is estimated that approximately 35% of patients in the USA infected with HIV are also infected with the hepatitis C virus, mainly because both viruses are blood-borne and present in similar populations. HCV is the leading cause of chronic liver disease in the United States. It has been demonstrated in clinical studies that HIV infection causes a more rapid progression of chronic hepatitis C to cirrhosis and liver failure.

For a detailed description of other infectious diseases and the various microbes that cause such disease see Mandell, Douglas and Bennett's Principles and Practice of Infectious Diseases—5TH edition, Churchill Livingstone, Inc., September 1998; Sexually Transmitted Diseases, Vol. 5 Gerald L. Mandell (Editor), Michael F. Rein (Editor), Churchill Livingstone, Inc., January 1996; Sexually Transmitted Diseases in Obstetrics and Gynecology, Sebastian Faro, Lippincott Williams & Wilkins, June 2001; or Sexually Transmitted Diseases, King K. Holmes, Per-Anders Mardh (Editor), Judith Wasserheit, McGraw-Hill, January 1999; each of which is incorporated herein by reference.

The present invention further provides methods for inhibiting a virus in or on an object. The object can be any of, but not limited to, a solution, a medical supply, or a medical equipment.

### Dialysis Filtration System

The methods of the invention encompass use of the compositions as described herein as part of a dialysis filtration system to remove infectious virus particles from patients.

For the treatment of a patient suffering from renal failure, various blood purifying methods have been proposed in which blood is taken out from the body of the patient to be purified and is then returned into the body.

Kidney dialysis machines are well known in the art and are illustrated, for example, in U.S. Pat. Nos. 3,598,727, 4,172,033, 4,267,040, and 4,769,134.

In certain examples, the dialysis system comprises a flow-through blood treatment device such as a hemodialyzer comprises a housing, a blood inlet, a blood outlet, and at least one membrane in the housing defining a blood flow path between the blood inlet and outlet on one side of the membrane, plus a second flow path defined on the other side of the membrane. Certain such devices are described in U.S. Pat. No. 5,643,190.

In one aspect, the invention features a method for elimination of a virus from the blood comprising contacting the blood with an effective amount of at least one of the following: (i) an isolated or purified antiviral protein comprising the amino acid sequence of SEQ ID NO: 1, an amino acid sequence that is about 90% or more identical to SEQ ID NO: 1, an amino acid sequence that is about 90% or more homologous to SEQ ID NO: 1, or a fragment thereof; (ii) an isolated or purified nucleic acid comprising a nucleotide sequence encoding the amino acid sequence of SEQ ID NO: 1; (iii) an isolated or purified antiviral protein comprising the amino acid sequence of SEQ ID NO: 2, an amino acid sequence that is about 90% or more identical to SEQ ID NO: 2, an amino acid sequence that is about 90% or more homologous to SEQ ID NO: 2, or a fragment thereof; (iv) an isolated or purified nucleic acid comprising a nucleotide sequence encoding the amino acid sequence of SEQ ID NO: 2; (v) an isolated or purified antiviral protein comprising the amino acid sequence of SEQ ID NO: 3, an amino acid sequence that is about 90% or more identical to SEQ ID NO: 3, an amino acid sequence that is about 90% or more homologous to SEQ ID NO: 3; (vi) an isolated or purified nucleic acid comprising a nucleotide sequence encoding the amino acid sequence of SEQ ID NO: 3, or a fragment thereof, and thereby eliminating the virus from the blood.

The methods of the invention can be used to remove infectious virus from contaminated blood or bodily fluids.

### Pharmaceutical Compositions

In yet another aspect of the invention, the compositions of the invention may be formulated as pharmaceutical compositions useful for the treatment, prevention or mitigation of infection by high-mannose enveloped viruses, for example HCV or HIV. “High mannose” is meant to refer to at least six, typically six to nine, linked mannose rings. High mannose envelope viruses are meant to include, but are not limited to HCV, HIV, influenza virus, measles virus, herpes virus 6, marburg virus, and ebola virus.

Also provided are methods for the treatment, prevention or mitigation of infection by such viruses, comprising administering a therapeutically or prophylactically effective amount of a pharmaceutical composition of the invention.

The pharmaceutical compositions of the invention may be administered or formulated with additional excipients, solvents, stabilizers, adjuvants, diluents, etc., depending upon the particular mode of administration and dosage form. The present protein variants and/or conjugates may be administered parenterally as well as non-parenterally. Specific administration routes include oral, ocular, vaginal, rectal, buccal, topical, nasal, ophthalmic, subcutaneous, intramuscular, intraveneous, intracerebral, transdermal, and pulmonary.

Pharmaceutical compositions of the invention generally comprise a therapeutically or prophylactically effective amount of the composition of the invention together with one or more pharmaceutically acceptable carriers. Formulations of the present invention, e.g., for parenteral administration, are most typically liquid solutions or suspensions. Generally, the pharmaceutical compositions for parenteral administration will be formulated in a non-toxic, inert, pharmaceutically acceptable aqueous carrier medium, preferably at a pH of about 5 to 8, more preferably 6 to 8. Inhalable formulations for pulmonary administration are generally liquids or powders, with powder formulations being generally preferred. Pharmaceutical compositions of the invention can also be formulated as a lyophilized solid which is reconstituted with a physiologically appropriate solvent prior to administration. Additional albeit less preferred compositions of the proteins and/or protein-polymer conjugates of the invention include syrups, creams, ointments, tablets, and the like.

The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which may be administered without undue toxicity. Suitable carriers may be large, slowly metabolized macromolecules such as proteins, polysaccharides, polylactic acids, polyglycolic acids, polymeric amino acids, amino acid copolymers, and inactive virus particles. Pharmaceutically acceptable carriers are determined in part by the particular composition being administered, as well as by the particular method used to administer the composition. Accordingly, there is a wide variety of suitable formulations of pharmaceutical compositions of the present invention (see, e.g., Remington's Pharmaceutical Sciences, 17th ed. 1985).

Pharmaceutically acceptable carriers in therapeutic compositions may contain liquids such as water, saline, glycerol and ethanol. Additionally, auxiliary substances, such as wetting or emulsifying agents, pH buffering substances, and the like, may be present in such vehicles. Typically, pharmaceutical compositions are prepared as injectables, either as liquid solutions or suspensions; solid forms suitable for solution in, or suspension in, liquid vehicles prior to injection may also be prepared. Liposomes are included within the definition of a pharmaceutically acceptable carrier.

The term “therapeutically or prophylactically effective amount” as used herein refers to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms, such as decreased body temperature. The precise effective amount for a subject will depend upon the subject's size and health, the nature and extent of the condition, and the therapeutics or combination of therapeutics selected for administration. Thus, it is not useful to specify an exact effective amount in advance. However, the effective amount for a given situation can be determined by routine experimentation and is within the judgement of the clinician.

For any compound, the therapeutically effective dose can be estimated initially either in cell culture assays, e.g., of cells infected with HCV, or in animal models, usually mice, rabbits, dogs, or pigs. The animal model may also be used to determine the appropriate concentration range and route of administration. Such information can then be used to determine useful doses and routes for administration in humans.

A therapeutically effective dose refers to that amount of active ingredient, for example, a composition of the invention as described herein, which ameliorates the symptoms or condition, or provides protection against infection.

Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, ED50/LD50. Pharmaceutical compositions which exhibit large therapeutic indices are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage form employed, sensitivity of the patient, and the route of administration.

The exact dosage will be determined by the practitioner, and will be determined and adjusted to provide sufficient levels of the composition or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

Normal dosage amounts may vary from 0.1 to 100 μg, up to a total dose of about 1 g, depending upon the route of administration. Guidance as to particular dosages and methods of delivery is provided in the literature and generally available to practitioners in the art.

In certain preferred examples, the compositions of the invention are administered systemically. In preferred examples, the compositions are preferably administered parenterally, e.g. by intramuscular or intravenous injection, thus avoiding the GI tract. Other modes of administration include transdermal and transmucosal administrations provided by patches and/or topical cream compositions. Transmucosal administrations can also include nasal spray formulations which include the proteins of the invention within a nasal formulation which contacts the nasal membranes and diffuses through those membranes directly into the cardiovascular system. Aerosol formulations for intrapulmonary delivery can also be used.

The compositions of the invention as described herein can also be included in devices for fixation or delivery of the composition to a site of interest. Such devices can include particles, magnetic beads, flow-through matrices, condoms, diaphragms, cervical caps, vaginal rings, sponges, foams, and gels. More particularly, the compositions of the invention can be covalently attached to the surface of a device via hydrolytically stable or unstable linkages. Alternatively, the compositions of the invention can be incorporated into the mechanical device, such as through the formation of foams and gels which utilize the compositions as an integral part of its core structure. Such devices can then be used in their ordinary manner to fix the variants and/or conjugates to a specific location or to deliver the variants and/or conjugates of the invention to a desired location.

The composition and formulations of this invention are useful for treating and preventing viral infections caused by HCV.

Suitable formulations can include, but are not limited to, creams, gels, foams, ointments, lotions, balms, waxes, salves, solutions, suspensions, dispersions, water in oil or oil in water emulsions, microemulsions, pastes, powders, oils, lozenges, boluses, and sprays, and the like.

In preferred embodiments, the compositions are creams, gels or ointments.

In certain preferred examples, such compositions adhere well to bodily tissues (i.e., mammalian tissues such as skin and mucosal tissue) and thus are very effective topically. Thus, the present invention provides a wide variety of uses of the compositions. Particularly preferred methods involve topical application, particularly to mucous membranes and skin, for example in oral, nasal, or vaginal cavities.

Compositions described herein can be used to provide effective topical antiviral activity and thereby treat and/or prevent HCV.

Compositions described herein can be used to provide effective topical antiviral or antimicrobial activity and thereby treat and/or prevent a wide variety of afflictions. Compositions described herein can be used for the prevention and/or treatment of one or more microorganism-caused infections or other afflictions. Compositions described herein can be used to provide effective topical antimicrobial activity and thereby treat and/or prevent a wide variety of afflictions. For example, they can be used in the treatment and/or prevention of afflictions that are caused, or aggravated by, microorganisms (e.g., Gram positive bacteria, Gram negative bacteria, fungi, protozoa, mycoplasma, yeast, enveloped viruses) on skin and/or mucous membranes, such as those in the nose, mouth, or other similar tissues.

In certain embodiments, the compositions of the invention may reduce the viral load at the infection site.

In other certain embodiments, the compositions that include creams, gels, foams, ointments, lotions, balms, waxes, salves, solutions, suspensions, dispersions, water in oil or oil in water emulsions, microemulsions, pastes, powders, oils, lozenges, boluses, and sprays, and the like include other agents.

The compositions may include other therapeutic agents.

Thus, for example, the compositions may contain additional compatible pharmaceutically active materials for combination therapy (such as supplementary antimicrobials, anti-parasitic agents, antipruritics, astringents, healing promoting agents, steroids, non-steroidal anti-inflammatory agents, or other anti-inflammatory agents), or may contain materials useful in physically formulating various dosage forms of the present invention, such as excipients, dyes, pigments, perfumes, fragrances, lubricants, thickening agents, stabilizers, skin penetration enhancers, preservatives, film forming polymers, or antioxidants. The compositions may also contain vitamins such as vitamin B, vitamin C, vitamin E, vitamin A, and derivates thereof.

It will also be appreciated that additional antiseptics, disinfectants, antiviral agents, or antibiotics may be included and are contemplated.

The compositions may include a penetration agent. A penetration agent is a compound that enhances the antiseptic diffusion into or through the skin or mucosal tissue by increasing the permeability of the tissue to the antimicrobial component and pharmacologically active agent, if present, to increase the rate at which the drug diffuses into or through the tissue. Examples of penetration agents are described in PCT Patent Application No. US 2006/008953.

In general, the gel, cream or ointment compositions may be, but not limited to, the following:

A hydrophobic or hydrophilic ointment: The compositions are formulated with a hydrophobic base (e.g., petrolatum, thickened or gelled water insoluble oils, and the like) and optionally having a minor amount of a water soluble phase. Hydrophilic ointments generally contain one or more surfactants or wetting agents.

The hydrophobic ointment is an anhydrous or nearly anhydrous formulation with a hydrophobic vehicle. Typically the components of the ointment are chosen to provide a semi-solid consistency at room temperature which softens or melts at skin temperature to aid in spreading. Suitable components to accomplish this include low to moderate amounts of natural and synthetic waxes, for example beeswax, carnuba wax, candelilla wax, ceresine, ozokerite, microcrystalline waxes, and paraffins. Viscous semi-crystalline materials such as petrolatum and lanolin are useful in higher amounts. The viscosity of the ointment can also be adjusted with oil phase thickeners including hydrophobically modified clays.

In certain preferred embodiments of the present invention, the compositions are chosen to spread easily and absorb relatively rapidly into the epidermis.

An oil-in-water emulsion: The compositions may be formulations in which the antiviral lipid component is emulsified into an emulsion comprising a discrete phase of a hydrophobic component and a continuous aqueous phase that includes water and optionally one or more polar hydrophilic material(s) as well as salts, surfactants, emulsifiers, and other components. These emulsions may include water-soluble or water-swellable polymers as well as one or more emulsifier(s) that help to stabilize the emulsion. These emulsions generally have higher conductivity values, as described in U.S. Pat. No. 7,030,203.

A water-in-oil emulsion: The compositions may be formulations in which the antiviral lipid component is incorporated into an emulsion that includes a continuous phase of a hydrophobic component and an aqueous phase that includes water and optionally one or more polar hydrophilic material(s) as well as salts or other components. These emulsions may include oil-soluble or oil-swellable polymers as well as one or more emulsifier(s) that help to stabilize the emulsion.

Thickened Aqueous gels: These systems include an aqueous phase which has been thickened by suitable natural, modified natural, or synthetic polymers as described below. Alternatively, the thickened aqueous gels can be thickened using suitable polyethoxylated alkyl chain surfactants that effectively thicken the composition as well as other nonionic, cationic, or anionic emulsifier systems. Preferably, cationic or anionic emulsifier systems are chosen since some polyethoxylated emulsifiers can inactivate the antiviral lipids especially at higher concentrations.

Hydrophilic gels: These are systems in which the continuous phase includes at least one water soluble or water dispersible hydrophilic component other than water. The formulations may optionally also contain water up to 20% by weight. Higher levels may be suitable in some compositions. Suitable hydrophilic components include one or more glycols such as polyols such as glycerin, propylene glycol, butylene glycols, etc., polyethylene glycols (PEG), random or block copolymers of ethylene oxide, propylene oxide, and/or butylene oxide, polyalkoxylated surfactants having one or more hydrophobic moieties per molecule, silicone copolyols, as well as combinations thereof, and the like. One skilled in the art will recognize that the level of ethoxylation should be sufficient to render the hydrophilic component water soluble or water dispersible at 23 C. In most embodiments, the water content is less than 20%, preferably less than 10%, and more preferably less than 5% by weight of the composition.

Compositions of the present invention optionally can include one or more surfactants to emulsify the composition and to help wet the surface and/or to aid in contacting the microorganisms. As used herein the term “surfactant” means an amphiphile (a molecule possessing both polar and nonpolar regions which are covalently bound) capable of reducing the surface tension of water and/or the interfacial tension between water and an immiscible liquid. The term is meant to include soaps, detergents, emulsifiers, surface active agents, and the like. The surfactant can be cationic, anionic, nonionic, or amphoteric. In preferred embodiments, the surfactant includes poloxamer, ethoxylated stearates, sorbitan oleates, high molecular weight crosslinked copolymers of acrylic acid and a hydrophobic comonomer, and cetyl and stearyl alcohols as cosurfactants.

A wide variety of conventional surfactants can be used; however, certain ethoxylated surfactants can reduce or eliminate the antimicrobial efficacy of the antiviral lipid component. The exact mechanism of this is not known and not all ethoxylated surfactants display this negative effect. For example, poloxamer (polyethylene oxide/polypropylene oxide) surfactants have been shown to be compatible with the antiviral lipid component, but ethoxylated sorbitan fatty acid esters such as those sold under the trade name TWEEN by ICI have not been compatible. It should be noted that these are broad generalizations and the activity could be formulation dependent. One skilled in the art can easily determine compatibility of a surfactant by making the formulation and testing for antimicrobial activity as described in U.S. Patent Publication No. 2005/0089539-A1. Combinations of various surfactants can be used if desired.

It should be noted that certain antiviral lipid components are amphiphiles and may be surface active. For example, certain antiviral alkyl monoglycerides described herein are surface active. For embodiments containing both an antiviral lipid component and a surfactant, the antiviral lipid component is considered distinct from a “surfactant” component.

For certain applications, it may be desirable to formulate the antiviral lipid in compositions that are thickened with soluble, swellable, or insoluble organic polymeric thickeners such as natural and synthetic polymers including polyacrylic acids, poly(N-vinyl pyrrolidones), cellulosic derivatives, and xanthan or guar gums or inorganic thickeners such as silica, fumed silica, precipitated silica, silica aerogel and carbon black, and the like; other particle fillers such as calcium carbonate, magnesium carbonate, kaolin, talc, titanium dioxide, aluminum silicate, diatomaceous earth, ferric oxide and zinc oxide, clays, and the like; ceramic microspheres or glass microbubbles; ceramic microspheres such as those available under the tradenames “ZEOSPHERES” or “Z-LIGHT” from 3M Company, St. Paul, Minn. The above fillers can be used alone or in combination in the compositions described herein.

One skilled in the art may refer to general reference texts for detailed descriptions of known techniques discussed herein or equivalent techniques. These texts include Poly(ethylene glycol) Chemistry: Biotechnical and Biomedical Applications, Harris (ed.), Plenum Press, New York (1992); Wong, Chemistry of Protein Conjugation and Cross-Linking, CRC Press (1991); Ausubel et al., Current Protocols in Molecular Biology, John Wiley and Sons, Inc. (1995); Sambrook et al., Molecular Cloning, A Laboratory Manual (2d ed.), Cold Spring Harbor Press, Cold Spring Harbor, N.Y. (1989); Birren et al., Genome Analysis: A Laboratory Manual, volumes 1 through 4, Cold Spring Harbor Press, Cold Spring Harbor, N.Y. (1997-1999); Plant Molecular Biology: A Laboratory Manual, Clark (ed.), Springer, N.Y. (1997); Richards et al., Plant Breeding Systems (2d ed.), Chapman & Hall, The University Press, Cambridge (1997); and Maliga et al., Methods in Plant Molecular Biology, Cold Spring Harbor Press, Cold Spring Harbor, N.Y. (1995).

### Dosage and Administration

The compositions of the invention may be administered systemically. As used herein, the term “systemic administration” is meant to include in vivo systemic absorption or accumulation of drugs in the blood stream followed by distribution throughout the entire body. Administration routes which lead to systemic absorption include, without limitation: intravenous, subcutaneous, intraperitoneal, intranasal, inhalation, oral, intrapulmonary and intramuscular. Each of these administration routes expose the desired negatively charged polymers, for example, nucleic acids, to an accessible diseased tissue. The rate of entry of a drug into the circulation has been shown to be a function of molecular weight or size.

The compositions of the invention can be administered to cells by a variety of methods known to those of skill in the art, including, but not restricted to, encapsulation in liposomes; by iontophoresis; or by incorporation into other vehicles, such as hydrogels, cyclodextrins, biodegradable nanocapsules, and bioadhesive microspheres; or by proteinaceous vectors (O'Hare and Normand, International PCT Publication No. WO 00/53722).

Alternatively, the nucleic acid/vehicle combination may be locally delivered by direct injection or by use of an infusion pump. Direct injection of the complexes of the invention, whether subcutaneous, intramuscular, or intradermal, can take place using standard needle and syringe methodologies, or by needle-free technologies such as those described in Conry, et al., Clin. Cancer Res. 5:2330-2337, 1999, and Barry, et al., International PCT Publication No. WO 99/31262.

The invention also features the use of the composition comprising surface-modified liposomes containing poly(ethylene glycol) lipids (PEG-modified, or long-circulating liposomes or stealth liposomes). These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug. Lasic, et al., Chem. Rev. 95:2601-2627, 1995; Ishiwata, et al., Chem. Pharm. Bull. 43:1005-1011, 1995. Such liposomes have been shown to accumulate selectively in tumors, presumably by extravasation and capture in the neovascularized target tissues. Lasic, et al., Science 267:1275-1276, 1995; Oku, et al., Biochim. Biophys. Acta 1238:86-90, 1995. The long-circulating liposomes enhance the pharmacokinetics and pharmacodynamics of DNA and RNA, particularly compared to conventional cationic liposomes which are known to accumulate in tissues of the MPS. Liu, et al., J. Biol. Chem. 42:24864-24870, 1995; Choi, et al., International PCT Publication No. WO 96/10391; Ansell, et al., International PCT Publication No. WO 96/10390; and Holland, et al., International PCT Publication No. WO 96/10392. Long-circulating liposomes are also likely to protect drugs from nuclease degradation to a greater extent compared to cationic liposomes, nucleotided on their ability to avoid accumulation in metabolically aggressive MPS tissues such as the liver and spleen.

For application to skin or mucosal tissue, for example, the compositions may be applied directly to the tissue from a collapsible container such as a flexible tube, blow/fill/seal container, pouch, capsule, etc. In this embodiment, the primary container itself is used to dispense the composition directly onto the tissue or it can be used to dispense the composition onto a separate applicator. Other application devices may also be suitable including applicators with foam tips, brushes, and the like. Importantly, the applicator must be able to deliver the requisite amount of composition to the tissue.

The compositions of the present invention can be delivered from various substrates for delivery to the tissue. For example, the compositions can be delivered from a wipe or pad which when contacted to tissue will deliver at least a portion of the composition to the tissue.

The present disclosure also includes compositions prepared for storage or administration, which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co., A. R. Gennaro ed., 1985. For example, preservatives, stabilizers, dyes and flavoring agents may be provided. These include sodium benzoate, sorbic acid and esters of p-hydroxybenzoic acid. In addition, antioxidants and suspending agents may be used.

A pharmaceutically effective dose is that dose required to prevent, inhibit the occurrence of, or treat (alleviate a symptom to some extent, preferably all of the symptoms) a disease state. In certain examples, the disease state may be cancer. The pharmaceutically effective dose depends on the type of disease, the composition used, the route of administration, the type of mammal being treated, the physical characteristics of the specific mammal under consideration, concurrent medication, and other factors that those skilled in the medical arts will recognize. Generally, an amount between 0.1 mg/kg and 100 mg/kg body weight/day of active ingredients is administered dependent upon potency of the negatively charged polymer.

### Patient Monitoring

The disease state or treatment of a patient having a disease or disorder, for example a neoplasia, can be monitored using the methods and compositions of the invention.

In one embodiment, the tumor progression of a patient can be monitored using the methods and compositions of the invention. Such monitoring may be useful, for example, in assessing the efficacy of a particular drug in a patient. For examples, therapeutics that alter the expression of a target polypeptide that is overexpressed in a neoplasia are taken as particularly useful in the invention.

### EXAMPLES

It is a novel finding of the instant invention that the antiviral proteins scytovirin (SVN) and griffithsin (GRFT) have (nanomolar) activity against the Hepatitis C virus (HCV).

This invention is further illustrated by the following examples, which should not be construed as limiting. All documents mentioned herein are incorporated herein by reference.

### Example 1

The invention features, generally, compositions and methods for treating viral infections, for example Hepatitis C virus (HCV) and Human Immunodeficiency Virus (HIV). There are six major genotypes of the HCV, which are indicated numerically (e.g., genotype 1-genotype 6); however, the invention is not intended to be limited to a single HCV strain.

Subgenomic replicon assays were performed to determine the activity of cyanovirin (CV-N), scytovirin (SVN), or griffithsin (GRFT) against HCV.

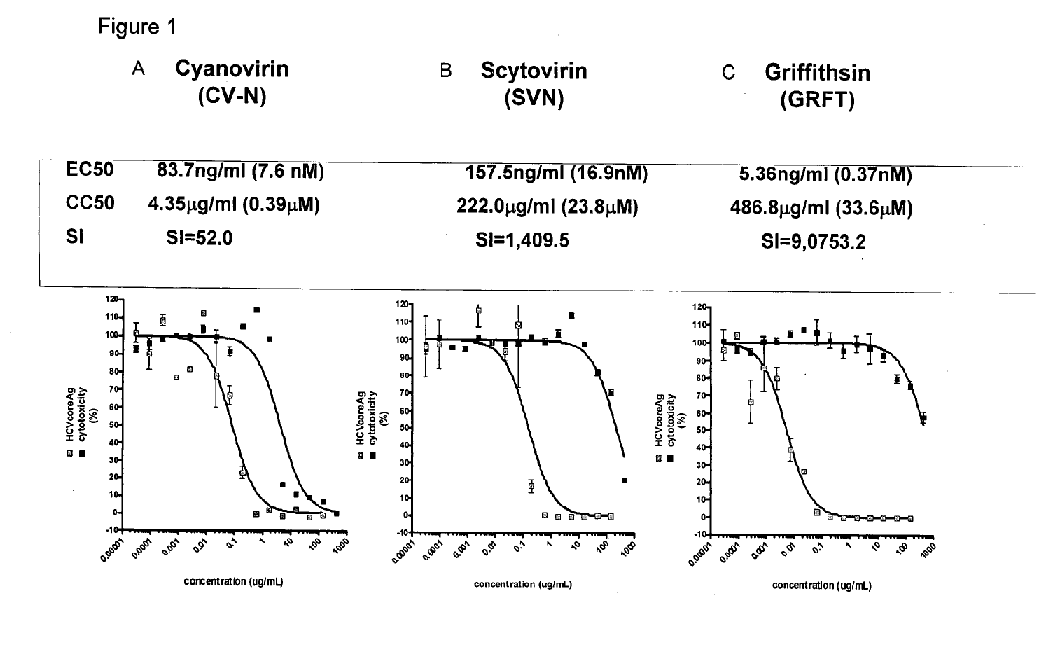

Results are shown in FIG. 1 (A-C). FIG. 1 shows a panel of three graphs showing the activity of cyanovirin (A), scytovirin (B), or griffithsin (C) against HCV. The molecular weight of the compounds were as follows: Cyanovirin, 11,009 Da (Native); Scytovirin, 9317 Da (Native); Griffithsin 14496 Da (His-tagged). In the experiments, HCV JFH-1 (genotype 2a) was used. Samples were diluted in water or PBS. Huh7.5.1 cells were treated with CV-N, SVN or GRFT at the indicated concentrations (μg/mL). At 72 hours poi HCV output was determined to measure anti-HCV efficacy of the compounds at the indicated concentrations. At 72 hours poi, WST cell proliferation assay (based on the reduction of tetrazolium salt WST-1 to soluble formazan by electron transport across the plasma membrane of dividing cells) was used to evaluate cytotoxicity of the compounds at the indicated concentrations.

As shown in FIG. 1A-C, CN-V demonstrated an EC-50 in the sub μg/mL order, but a higher cytotoxicity than SVN and GRFT. SVN and GRFT show low cytotoxicity and good SI, and an EC-50 in the μg/mL order. Overall, SVN shows modestly high activity, and GRFT showed considerably high (the highest activity) in the experiments described herein.

The experiments described herein demonstrate an anti-HCV activity of GRFT at a nanomolar or subnanomolar level (see, e.g. FIG. 1). Because of the biological nature of these compounds (i.e. that they are carbohydrate binding proteins), the potent anti-HCV activity of GRFT that is observed may be due to the inhibition of HCV entry or the HCV attachment process, not to the inhibition of post-entry replication mechanism. Further, it may be thought that the inhibition of viral entry or the attachment process cannot be evaluated by the subgenomic replicon assay, as has been previously described.

It is possible that GRFT binds additional targets, and as such this promiscuity in binding accounts for its lower activity in the described experiments. Additional targets may include proteins glycosylated with high mannose oligosaccharides.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method of treating or preventing a viral infection in a subject comprising:
administering to the subject an effective amount of one or more of the following: (i) an isolated or purified antiviral protein comprising the amino acid sequence of SEQ ID NO: 1, an amino acid sequence that is about 90% or more identical to SEQ ID NO: 1, an amino acid sequence that is about 90% or more homologous to SEQ ID NO: 1, or a fragment thereof; (ii) an isolated or purified nucleic acid comprising a nucleotide sequence encoding the amino acid sequence of SEQ ID NO: 1; (iii) an isolated or purified antiviral protein comprising the amino acid sequence of SEQ ID NO: 2, an amino acid sequence that is about 90% or more identical to SEQ ID NO: 2, an amino acid sequence that is about 90% or more homologous to SEQ ID NO: 2, or a fragment thereof; (iv) an isolated or purified nucleic acid comprising a nucleotide sequence encoding the amino acid sequence of SEQ ID NO: 2; (v) an isolated or purified antiviral protein comprising the amino acid sequence of SEQ ID NO: 3, an amino acid sequence that is about 90% or more identical to SEQ ID NO: 3, an amino acid sequence that is about 90% or more homologous to SEQ ID NO: 3; (vi) an isolated or purified nucleic acid comprising a nucleotide sequence encoding the amino acid sequence of SEQ ID NO: 3, or a fragment thereof,
thereby treating or preventing the viral infection in a subject.

2. The method of claim 1, wherein the viral infection is caused by a virus with a coat protein comprising high-mannose oligosaccharides.

3. The method of claim 2, wherein the virus is hepatitis C virus (HCV).

4. The method of claim 2, wherein the virus is human immunodeficiency virus (HIV).

5. The method of claim 1, further comprising a variant of (i) (ii) or (iii), wherein the variant comprises one or more conservative or neutral amino acid substitutions or one or more amino acid additions at the N-terminus or C-terminus, wherein the variant has antiviral activity characteristic of the antiviral protein consisting essentially of the amino acid sequence of SEQ ID NO: 1, SEQ ID NO: 2 or SEQ ID NO: 3.

6. The method of claim 1, further comprising a fusion protein of (i) (ii) or (iii) and at least one effector component, wherein the fusion protein has antiviral activity characteristic of the antiviral protein consisting essentially of the amino acid sequence of SEQ ID NO: 1, SEQ ID NO: 2 or SEQ ID NO: 3.

7. The method of claim 6, wherein the fusion protein comprises albumin.

8. The method of claim 1, wherein the nucleic acid comprising a nucleotide sequence encoding the amino acid sequence of SEQ ID NO: 1, SEQ ID NO: 2 or SEQ ID NO: 3 is contained in a vector.

9-13. (canceled)

14. A method of inhibiting a virus in a biological sample comprising:
contacting the biological sample with an effective amount of one or more of the following: (i) an isolated or purified antiviral protein comprising the amino acid sequence of SEQ ID NO: 1, an amino acid sequence that is about 90% or more identical to SEQ ID NO: 1, an amino acid sequence that is about 90% or more homologous to SEQ ID NO: 1, or a fragment thereof; (ii) an isolated or purified nucleic acid comprising a nucleotide sequence encoding the amino acid sequence of SEQ ID NO: 1; (iii) an isolated or purified antiviral protein comprising the amino acid sequence of SEQ ID NO: 2, an amino acid sequence that is about 90% or more identical to SEQ ID NO: 2, an amino acid sequence that is about 90% or more homologous to SEQ ID NO: 2, or a fragment thereof; (iv) an isolated or purified nucleic acid comprising a nucleotide sequence encoding the amino acid sequence of SEQ ID NO: 2; (v) an isolated or purified antiviral protein comprising the amino acid sequence of SEQ ID NO: 3, an amino acid sequence that is about 90% or more identical to SEQ ID NO: 3, an amino acid sequence that is about 90% or more homologous to SEQ ID NO: 3; (vi) an isolated or purified nucleic acid comprising a nucleotide sequence encoding the amino acid sequence of SEQ ID NO: 3, or a fragment thereof,
thereby inhibiting the virus in the biological sample.

15. A method of treating or preventing a viral infection caused by a virus in or on the skin or mucous membrane comprising:
contacting the affected area with a topical composition comprising an effective amount of one or more of the following: (i) an isolated or purified antiviral protein comprising the amino acid sequence of SEQ ID NO: 1, an amino acid sequence that is about 90% or more identical to SEQ ID NO: 1, an amino acid sequence that is about 90% or more homologous to SEQ ID NO: 1, or a fragment thereof; (ii) an isolated or purified nucleic acid comprising a nucleotide sequence encoding the amino acid sequence of SEQ ID NO: 1; (iii) an isolated or purified antiviral protein comprising the amino acid sequence of SEQ ID NO: 2, an amino acid sequence that is about 90% or more identical to SEQ ID NO: 2, an amino acid sequence that is about 90% or more homologous to SEQ ID NO: 2, or a fragment thereof; (iv) an isolated or purified nucleic acid comprising a nucleotide sequence encoding the amino acid sequence of SEQ ID NO: 2; (v) an isolated or purified antiviral protein comprising the amino acid sequence of SEQ ID NO: 3, an amino acid sequence that is about 90% or more identical to SEQ ID NO: 3, an amino acid sequence that is about 90% or more homologous to SEQ ID NO: 3; (vi) an isolated or purified nucleic acid comprising a nucleotide sequence encoding the amino acid sequence of SEQ ID NO: 3, or a fragment thereof,
thereby treating or preventing a viral infection caused by a virus in or on the skin or mucous membrane.

16-18. (canceled)

19. A method of inhibiting a virus in or on an object comprising:
contacting the object with an effective amount of one or more of the following: (i) an isolated or purified antiviral protein comprising the amino acid sequence of SEQ ID NO: 1, an amino acid sequence that is about 90% or more identical to SEQ ID NO: 1, an amino acid sequence that is about 90% or more homologous to SEQ ID NO: 1, or a fragment thereof; (ii) an isolated or purified nucleic acid comprising a nucleotide sequence encoding the amino acid sequence of SEQ ID NO: 1; (iii) an isolated or purified antiviral protein comprising the amino acid sequence of SEQ ID NO: 2, an amino acid sequence that is about 90% or more identical to SEQ ID NO: 2, an amino acid sequence that is about 90% or more homologous to SEQ ID NO: 2, or a fragment thereof; (iv) an isolated or purified nucleic acid comprising a nucleotide sequence encoding the amino acid sequence of SEQ ID NO: 2; (v) an isolated or purified antiviral protein comprising the amino acid sequence of SEQ ID NO: 3, an amino acid sequence that is about 90% or more identical to SEQ ID NO: 3, an amino acid sequence that is about 90% or more homologous to SEQ ID NO: 3; (vi) an isolated or purified nucleic acid comprising a nucleotide sequence encoding the amino acid sequence of SEQ ID NO: 3, or a fragment thereof,
thereby inhibiting the virus in or on the object, or
A method for elimination of a virus from the blood of a subject comprising:
contacting the blood with an effective amount of one or more of the following: (i) an isolated or purified antiviral protein comprising the amino acid sequence of SEQ ID NO: 1, an amino acid sequence that is about 90% or more identical to SEQ ID NO: 1, an amino acid sequence that is about 90% or more homologous to SEQ ID NO: 1, or a fragment thereof; (ii) an isolated or purified nucleic acid comprising a nucleotide sequence encoding the amino acid sequence of SEQ ID NO: 1; (iii) an isolated or purified antiviral protein comprising the amino acid sequence of SEQ ID NO: 2, an amino acid sequence that is about 90% or more identical to SEQ ID NO: 2, an amino acid sequence that is about 90% or more homologous to SEQ ID NO: 2, or a fragment thereof; (iv) an isolated or purified nucleic acid comprising a nucleotide sequence encoding the amino acid sequence of SEQ ID NO: 2; (v) an isolated or purified antiviral protein comprising the amino acid sequence of SEQ ID NO: 3, an amino acid sequence that is about 90% or more identical to SEQ ID NO: 3, an amino acid sequence that is about 90% or more homologous to SEQ ID NO: 3; (vi) an isolated or purified nucleic acid comprising a nucleotide sequence encoding the amino acid sequence of SEQ ID NO: 3, or a fragment thereof,
thereby eliminating the virus from the blood.

20-30. (canceled)

31. A method of treating or preventing a viral infection in a subject comprising:
administering to the subject one or more antibodies selected from: (i) an antibody that binds a protein comprising the amino acid sequence of SEQ ID NO: 1; (ii) an antibody that binds a protein comprising the amino acid sequence of SEQ ID NO: 2; (iii) an antibody that binds a protein comprising the amino acid sequence of SEQ ID NO: 3, or a fragment thereof, in an amount sufficient to induce in the subject an immune response to the virus;
thereby treating or preventing the viral infection in a subject, or
A method of inhibiting a virus in a biological sample comprising:
administering to the subject one or more antibodies selected from: (i) an antibody that binds a protein comprising the amino acid sequence of SEQ ID NO: 1; (ii) an antibody that binds a protein comprising the amino acid sequence of SEQ ID NO: 2; (iii) an antibody that binds a protein comprising the amino acid sequence of SEQ ID NO: 3, or a fragment thereof, in an amount sufficient to induce in the subject an immune response to the virus;
thereby inhibiting the virus in a biological sample.

32. The method of claim 31, wherein the viral infection is caused by a virus with a coat protein comprising high-mannose oligosaccharides.

33. The method of claim 31, wherein the virus is hepatitis C virus (HCV).

34. The method of claim 31, wherein the virus is human immunodeficiency virus (HIV).

35-36. (canceled)

37. A method of inhibiting a virus in a biological sample comprising:
administering to the subject one or more antibodies selected from: (i) an antibody that binds a protein comprising the amino acid sequence of SEQ ID NO: 1; (ii) an antibody that binds a protein comprising the amino acid sequence of SEQ ID NO: 2; (iii) an antibody that binds a protein comprising the amino acid sequence of SEQ ID NO: 3, or a fragment thereof, in an amount sufficient to induce in the subject an immune response to the virus;
thereby inhibiting the virus in a biological sample, or
A method for elimination of a virus from the blood of a subject comprising:
administering to the subject one or more antibodies selected from: (i) an antibody that binds a protein comprising the amino acid sequence of SEQ ID NO: 1; (ii) an antibody that binds a protein comprising the amino acid sequence of SEQ ID NO: 2; (iii) an antibody that binds a protein comprising the amino acid sequence of SEQ ID NO: 3, or a fragment thereof, in an amount sufficient to induce in the subject an immune response to the virus;
thereby eliminating the virus from the blood.

38. The method of claim 37, wherein the biological sample is selected from the group consisting of: blood, a blood product, cells, a tissue, an organ, sperm, a vaccine formulation, and a bodily fluid.

39. (canceled)

40. The method of claim 39, wherein the blood is from a blood transfusion.

41. A method of treating or preventing a viral infection caused by a virus in or on the skin or mucous membrane comprising:
administering to the subject one or more antibodies selected from: (i) an antibody that binds a protein comprising the amino acid sequence of SEQ ID NO: 1; (ii) an antibody that binds a protein comprising the amino acid sequence of SEQ ID NO: 2; (iii) an antibody that binds a protein comprising the amino acid sequence of SEQ ID NO: 3, or a fragment thereof, in an amount sufficient to induce in the subject an immune response to the virus;
thereby treating or preventing a viral infection caused by a virus in or on the skin or mucous membrane, or
A method of inhibiting a virus in or on an object comprising:
administering to the subject one or more antibodies selected from: (i) an antibody that binds a protein comprising the amino acid sequence of SEQ ID NO: 1; (ii) an antibody that binds a protein comprising the amino acid sequence of SEQ ID NO: 2; (iii) an antibody that binds a protein comprising the amino acid sequence of SEQ ID NO: 3, or a fragment thereof, in an amount sufficient to induce in the subject an immune response to the virus;
thereby inhibiting the virus in or on an object.

42-49. (canceled)


SEQUENCE LISTING (5 sequences)

SEQ ID NO: 1 - protein, 95 aa, Scytonema varium
GSGPTYCWNEANNPGGPNRCSNNKQCDGARTCSSSGFCQGTSRKPDPGPKGPTYCWDEAKNPGGPNRCSNSKQCDGARTCSSSGFCQGTAGHAAA

SEQ ID NO: 2 - protein, 101 aa, Nostoc ellipsosporum
LGKFSQTCYNSAIQGSVLTSTCERTNGGYNTSSIDLNSVIENVDGSLKWQPSNFIETCRNTQLAGSSELAAECKTRAQQFVSTKINLDDHIANIDGTLKYE

SEQ ID NO: 3 - protein, 121 aa, Griffithsia sp.
  MOD_RES (31)..(31): Any amino acid
SLTHRKFGGSGGSPFSGLSSIAVRSGSYLDXIIIDGVHHGGSGGNLSPTFTFGSGEYISNMTIRSGDYIDNISFETNMGRRFGPYGGSGGSANTLSNVKVIQINGSAGDYLDSLDIYYEQY

SEQ ID NO: 4 - protein, 6 aa, Artificial Sequence
  source: /note=“Description of Artificial Sequence 6xHis tag”
HHHHHH

SEQ ID NO: 5 - protein, 5 aa, Griffithsia sp.
GGSGG